SEQUENCE LISTING (23 sequences)

SEQ ID NO: 1 - protein, 73 aa, Human
DREQAPNLVYMVTGNPASDEIKRLPGDIQVVPIGVGPNANVQELERIGWPNAPILIQDFETLPREAPDLVLQR

SEQ ID NO: 2 - protein, 188 aa, Human
GLLGVSTLGPKRNSMVLDVAFVLEGSDKIGEADFNRSKEFMEEVIQRMDVGQDSIHVTVLQYSYMVTVEYPFSEAQSKGDILQRVREIRYQGGNRTNTGLALRYLSDHSFLVSQGDREQAPNLVYMVTGNPASDEIKRLPGDIQVVPIGVGPNANVQELERIGWPNAPILIQDFETLPREAPDLVLQR

SEQ ID NO: 3 - protein, 1424 aa, Human
MHQRHPRARCPPLCVAGILACGFLLGCWGPSHFQQSCLQALEPQAVSSYLSPGAPLKGRPPSPGFQRQRQQRRAAGGILHLELLVAVGPDVFQAHQEDTERYVLTNLNIGAELLRDPSLGAQFRVHLVKMVILTEPEGAPNITANLTSSLLSVCGWSQTINPEDDTDPGHADLVLYITRFDLELPDGNRQVRGVTQLGGACSPTWSCLITEDTGFDLGVTIAHEIGHSFGLEHDGAPGSGCGPSGHVMASDGAAPRAGLAWSPCSRRQLLSLLSAGRARCVWDPPRPQPGSAGHPPDAQPGLYYSANEQCRVAFGPKAVACTFAREHLDMCQALSCHTDPLDQSSCSRLLVPLLDGTECGVEKWCSKGRCRSLVELTPIAAVHGRWSSWGPRSPCSRSCGGGVVTRRRQCNNPRPAFGGACVGADLQAEMCNTQACEKTQLEFMSQQCARTDGQPLRSSPGGASFYHWGAAVPHSQGDALCRHMCRAIGESFIMKRGDSFLDGTRCMPSGPREDGTLSLCVSGSCRTFGCDGRMDSQQVWDRCQVCGGDNSTCSPRKGSFTAGRAREYVTFLTVTPNLTSVYIANHRPLFTHLAVRIGGRYVVAGKMSISPNTTYPSLLEDGRVEYRVALTEDRLPRLEEIRIWGPLQEDADIQVYRRYGEEYGNLTRPDITFTYFQPKPRQAWVWAAVRGPCSVSCGAGLRWVNYSCLDQARKELVETVQCQGSQQPPAWPEACVLEPCPPYWAVGDFGPCSASCGGGLRERPVRCVAQGSLLKTLPPARCRAGAQQPAVALETCNPQPCPARWEVSEPSSCTSAGGAGLALENETCVPGADGLEAPVTEGPGSVDEKLPAPEPCVGMSCPPGWGHLDATSAGEKAPSPWGSIRTGAQAAHVWTPAAGSCSVSCGRGLMELRFLCMDSALRVPVQEELCGLASKPGSRREVCQAVPCPARWQYKLAACSVSCGRGVVRRILYCARAHGEDDGEEILLDTQCQGLPRPEPQEACSLEPCPPRWKVMSLGPCSASCGLGTARRSVACVQLDQGQDVEVDEAACAALVRPEASVPCLIADCTYRWHVGTWMECSVSCGDGIQRRRDTCLGPQAQAPVPADFCQHLPKPVTVRGCWAGPCVGQGTPSLVPHEEAAAPGRTTATPAGASLEWSQARGLLFSPAPQPRRLLPGPQENSVQSSACGRQHLEPTGTIDMRGPGQADCAVAIGRPLGEVVTLRVLESSLNCSAGDMLLLWGRLTWRKMCRKLLDMTFSSKTNTLVVRQRCGRPGGGVLLRYGSQLAPETFYRECDMQLFGPWGEIVSPSLSPATSNAGGCRLFINVAPHARIAIHALATNMGAGTEGANASYILIRDTHSLRTTAFHGQQVLYWESESSQAEMEFSEGFLKAQASLRGQYWTLQSWVPEMQDPQSWKGKEGT

SEQ ID NO: 4 - protein, 1339 aa, Human
MHQRHPRARCPPLCVAGILACGFLLGCWGPSHFQQSCLQALEPQAVSSYLSPGAPLKGRPPSPGFQRQRQRQRRAAGGILHLELLVAVGPDVFQAHQEDTERYVLTNLNIGAELLRDPSLGAQFRVHLVKMVILTEPEGAPNITANLTSSLLSVCGWSQTINPEDDTDPGHADLVLYITRFDLELPDGNRQVRGVTQLGGACSPTWSCLITEDTGFDLGVTIAHEIGHSFGLEHDGAPGSGCGPSGHVMASDGAAPRAGLAWSPCSRRQLLSLLSANEQCRVAFGPKAVACTFAREHLDMCQALSCHTDPLDQSSCSRLLVPLLDGTECGVEKWCSKGRCRSLVELTPIAAVHGRWSSWGPRSPCSRSCGGGVVTRRRQCNNPRPAFGGRACVGADLQAEMCNTQACEKTQLEFMSQQCARTDGQPLRSSPGGASFYHWGAAVPHSQGDALCRHMCRAIGESFIMKRGDSFLDGTRCMPSGPREDGTLSLCVSGSCRTFGCDGRMDSQQVWDRCQVCGGDNSTCSPRKGSFTAGRAREYVTFLTVTPNLTSVYIANHRPLFTHLAVRIGGRYVVAGKMSISPNTTYPSLLEDGRVEYRVALTEDRLPRLEEIRIWGPLQEDADIQVYRRYGEEYGNLTRPDITFTYFQPKPRQAWVWAAVRGPCSVSCGAGLRWVNYSCLDQARKELVETVQCQGSQQPPAWPEACVLEPCPPYWAVGDFGPCSASCGGGLRERPVRCVEAQGSLLKTLPPARCRAGAQQPAVALETCNPQPCPARWEVSEPSSCTSAGGAGLALENETCVPGADGLEAPVTEGPGSVDEKLPAPEPCVGMSCPPGWGHLDATSAGEKAPSPWGSIRTGAQAAHVWTPAAGSCSVSCGRGLMELRFLCMDSALRVPVQEELCGLASKPGSRREVCQAVPCPARWQYKLAACSVSCGRGVVRRILYCARAHGEDDGEEILLDTQCQGLPRPEPQEACSLEPCPPRWKVMSLGPCSASCGLGTARRSVACVQLDQGQDVEVDEAACAALVRPEASVPCLIADCTYRWHVGTWMECSVSCGDGIQRRRDTCLGPQAQAPVPADFCQHLPKPVTVRGCWAGPCVGQGACGRQHLEPTGTIDMRGGQADCAVAIGRPLGEVVTLRVLESSLNCSAGDMLLLWGRLTWRKMCRKLLDMTFSSKTNTLVVRQRCGRPGGGVLLRYGSQLAPETFYRECDMQLFGPWGEIVSPSLSPATSNAGGCRLFINVAPHARIAIHALATNMGAGTEGANASYILIRDTHSLRTTAFHGQQVLYWESESSQAEMEFSEGFLKAQASLRGQYWTLQSWVPEMQDPQSWKGKEGT

SEQ ID NO: 5 - protein, 1368 aa, Human
MHQRHPRARCPPLCVAGILACGFLLGCWGPSHFQQSCLQALEPQAVSSYLSPGAPLKGRPPSPGFQRQRQRQRRAAGGILHLELLVAVGPDVFQAHQEDTERYVLTNLNIGAELLRDPSLGAQFRVHLVKMVILTEPEGAPNITANLTSSLLSVCGWSQTINPEDDTDPGHADLVLYITRFDLELPDGNRQVRGVTQLGGACSPTWSCLITEDTGFDLGVTIAHEIGHSFGLEHDGAPGSGCGPSGHVMASDGAAPRAGLAWSPCSRRQLLSLLSAGRARCVWDPPRPQPGSAGHPPDAQPGLYYSANEQCRVAFGPKAVACTFAREHLDMCQALSCHTDPLDQSSCSRLLVPLLDGTECGVEKWCSKGRCRSLVELTPIAAVHGRWSSWGPRSPCSRSCGGGVVTRRRQCNNPRPAFGGACVGADLQAEMCNTQACEKTQLEFMSQQCARTDGQPLRSSPGGASFYHWGAAVPHSQGDALCRHMCRAIGESFIMKRGDSFLDGTRCMPSGPREDGTLSLCVSGSCRTFGCDGRMDSQQVWDRCQVCGGDNSTCSPRKGSFTAGRAREYVTFLTVTPNLTSVYIANHRPLFTHLAVRIGGRYVVAGKMSISPNTTYPSLLEDGRVEYRVALTEDRLPRLEEIRIWGPLQEDADIQVYRRYGEEYGNLTRPDITFTYFQPKPRQAWVWAAVRGPCSVSCGAGLRWVNYSCLDQARKELVETVQCQGSQQPPAWPEACVLEPCPPYWAVGDFGPCSASCGGGLRERPVRCVAQGSLLKTLPPARCRAGAQQPAVALETCNPQPCPARWEVSEPSSCTSAGGAGLALENETCVPGADGLEAPVTEGPGSVDEKLPAPEPCVGMSCPPGWGHLDATSAGEKAPSPWGSIRTGAQAAHVWTPVAGSCSVSCGRGLMELRFLCMDSALRVPVQEELCGLASKPGSRREVCQAVPCPARWQYKLAACSVSCGRGVVRRILYCARAHGEDDGEEILLDTQCQGLPRPEPQEACSLEPCPPRWKVMSLGPCSASCGLGTARRSVACVQLDQGQDVEVDEAACAALVRPEASVPCLIADCTYRWHVGTWMECSVSCGDGIQRRRDTCLGPQAQAPVPADFCQHLPKPVVRGCWAGPCVGQGACGRQHLEPTGTIDMRGPGQADCAVAIGRPLGEVVTLRVLESSLNCSAGDMLLLWGRLTWRKMCRKLLDMTFSSKTNTLVVRQRCGRPGGGVLLRYGSQLAPETFYRECDMQLFGPWGEIVSPSLSPATSNAGGCRLFINVAPHARIAIHALATNMGAGTEGANASYILIRDTHSLRTTAFHGQQVLYWESESSQAEMEFSEGFLKAQASLRGQYWTLQSWVPEMQDPQSWKGKEGT

SEQ ID NO: 6 - protein, 344 aa, Human
MHQRHPRARCPPLCVAGILACGFLLGCWGPSHFQQSCLQALEPQAVSSYLSPGAPLKGRPPSPGFQRQRQRQRRAAGGILHLELLVAVGPDVFQAHQEDTERYVLTNLNIGAELLRDPSLGAQFRVHLVKMVILTEPEGAPNITANLTSSLLSVCGWSQTINPEDDTDPGHADLVLYITRFDLELPDGNRQVRGVTQLGGACSPTWSCLITEDTGFDLGVTIAHEIGHSFGLEHDGAPGSGCGPSGHVMASDGAAPRAGLAWSPCSRRQLLSLLRPVPPSPLPLLATHLCAGRSLSLGPSQEPAPGGGRGPRTPVPTHKRPRFQTLPSSCPLLRPASSRCTPRD

SEQ ID NO: 7 - DNA, 8922 bp, Human
agctcacagctattgtggtgggaaagggagggtggttggtggatgtcacagcttgggctttatctcccccagcagtggggactccacagcccctgggctacataacagcaagacagtccggagctgtagcagacctgattgagcctttgcagcagctgagagcatggcctagggtgggcggcaccattgtccagcagctgagtttcccagggaccttggagatagccgcagccctcatttgcaggggaaggcaccattgtccagcagctgagtttcccagggaccttggagatagccgcagccctcatttatgattcctgccagatttgccggggtgctgcttgctctggccctcattttgccagggaccctttgtgcagaaggaactcgcggcaggtcatccacggcccgatgcagccttttcggaagtgacttcgtcaacacctttgatgggagcatgtacagctttgcgggatactgcagttacctcctggcagggggctgccagaaacgctccttctcgattattggggacttccagaatggcaagagagtgagcctctccgtgtatcttggggaattttttgacatccatttgtttgtcaatggtaccgtgacacagggggaccaaagagtctccatgccctatgcctccaaagggctgtatctagaaactgaggctgggtactacaagctgtccggtgaggcctatggctttgtggccaggatcgatggcagcggcaactttcaagtcctgctgtcagacagatacttcaacaagacctgcgggctgtgtggcaactttaacatctttgctgaagatgactttatgacccaagaagggaccttgacctcggacccttatgactttgccaactcatgggctctgagcagtggagaacagtggtgtgaacgggcatctcctcccagcagctcatgcaacatctcctctggggaaatgcagaagggcctgtgggagcagtgccagcttctgaagagcacctcggtgtttgcccgctgccaccctctggtggaccccgagccttttgtggccctgtgtgagaagactttgtgtgagtgtgctggggggctggagtgcgcctgccctgccctcctggagtacgcccggacctgtgcccaggagggaatggtgctgtacggctggaccgaccacagcgcgtgcagcccagtgtgccctgctggtatggagtataggcagtgtgtgtccccttgcgccaggacctgccagagcctgcacataatgaaatgtgtcaggagcgatgcgtggatggctgcagctgccctgagggacagctcctggatgaaggcctctgcgtggagagcaccgagtgtccctgcgtgcattccggaaagcgctaccctcccggcacctccctctctcgagactgcaacacctgcatttgccgaaacagccagtggatctgcagcaatgaagaatgtccaggggagtgccttgtcactggtcaatcccacttcaagagctttgacaacagatacttcaccttcagtgggatctgccagtacctgctggcccgggattgccaggaccactccttctccattgtcattgagactgtccagtgtgctgatgaccgcgacgctgtgtgcacccgctccgtcaccgtccggctgcctggcctgcacaacagccttgtgaaactgaagcatggggcaggagttgccatggatggccaggacatccagctccccctcctgaaaggtgacctccgcatccagcatacagtgacggcctccgtgcgcctcagctacggggaggacctgcagatggactgggatggccgcgggaggctgctggtgaagctgtcccccgtctacgccgggaagacctgcggcctgtgtgggaattacaatggcaaccagggcgacgacttccttaccccctctgggctggcagagccccgggtggaggacttcgggaacgcctggaagctgcacggggactgccaggacctgcagaagcagcacagcgatccctgcgccctcaacccgcgcatgaccaggttctccgaggaggcgtgcgcggtcctgacgtcccccacattcgaggcctgccatcgtgccgtcagcccgctgccctacctgcggaactgccgctacgacgtgtgctcctgctcggacggccgcgagtgcctgtgcggcgccctggccagctatgccgcggcctgcgcggggagaggcgtgcgcgtcgcgtggcgcgagccaggccgctgtgagctgaactgcccgaaaggccaggtgtacctgcagtgcgggaccccctgcaacctgacctgccgctctctctcttacccggatgaggaatgcaatgaggcctgcctggagggctgcttctgccccccagggctctacatggatgagaggggggactgcgtgcccaaggcccagtgcccctgttactatgacggtgagatcttccagccagaagacatcttctcagaccatcacaccatgtgctactgtgaggatggcttcatgcactgtaccatgagtggagtccccggaagcttgctgcctgacgctgtcctcagcagtcccctgtctcatcgcagcaaaaggagcctatcctgtcggccccccatggtcaagctggtgtgtcccgctgacaacctgcgggctgaagggctcgagtgtaccaaaacgtgccagaactatgacctggagtgcatgagcatgggctgtgtctctggctgcctctgccccccgggcatggtccggcatgagaacagatgtgtggccctggaaaggtgtccctgcttccatcagggcaaggagtatgcccctggagaaacagtgaagattggctgcaacacttgtgtctgtcgggaccggaagtggaactgcacagaccatgtgtgtgatgccacgtgctccacgatcggcatggcccactacctcaccttcgacgggctcaaatacctgttccccggggagtgccagtacgttctggtgcaggattactgcggcagtaaccctgggacctttcggatcctagtggggaataagggatgcagccacccctcagtgaaatgcaagaaacgggtcaccatcctggtggagggaggagagattgagctgtttgacggggaggtgaatgtgaagaggcccatgaaggatgagactcactttgaggtggtggagtctggccggtacatcattctgctgctgggcaaagccctctccgtggtctgggaccgccacctgagcatctccgtggtcctgaagcagacataccaggagaaagtgtgtggcctgtgtgggaattttgatggcatccagaacaatgacctcaccagcagcaacctccaagtggaggaagaccctgtggactttgggaactcctggaaagtgagctcgcagtgtgctgacaccagaaaagtgcctctggactcatcccctgccacctgccataacaacatcatgaagcagacgatggtggattcctcctgtagaatccttaccagtgacgtcttccaggactgcaacaagctggtggaccccgagccatatctggatgtctgcatttacgacacctgctcctgtgagtccattggggactgcgcctgcttctgcgacaccattgctgcctatgcccacgtgtgtgcccagcatggcaaggtggtgacctggaggacggccacattgtgcccccagagctgcgaggagaggaatctccgggagaacgggtatgagtgtgagtggcgctataacagctgtgcacctgcctgtcaagtcacgtgtcagcaccctgagccactggcctgccctgtgcagtgtgtggagggctgccatgcccactgccctccagggaaaatcctggatgagcttttgcagacctgcgttgaccctgaagactgtccagtgtgtgaggtggctggccggcgttttgcctcaggaaagaaagtcaccttgaatcccagtgaccctgagcactgccagatttgccactgtgatgttgtcaacctcacctgtgaagcctgccaggagccgggaggcctggtggtgcctcccacagatgccccggtgagccccaccactctgtatgtggaggacatctcggaaccgccgttgcacgatttctactgcagcaggctactggacctggtcttcctgctggatggctcctccaggctgtccgaggctgagtttgaagtgctgaaggcctttgtggtggacatgatggagcggctgcgcatctcccagaagtgggtccgcgtggccgtggtggagtaccacgacggctcccacgcctacatcgggctcaaggaccggaagcgaccgtcagagctgcggcgcattgccagccaggtgaagtatgcgggcagccaggtggcctccaccagcgaggtcttgaaatacacactgttccaaatcttcagcaagatcgaccgccctgaagcctcccgcatcgccctgctcctgatggccagccaggagccccaacggatgtcccggaactttgtccgctacgtccagggcctgaagaagaagaaggtcattgtgatcccggtgggcattgggccccatgccaacctcaagcagatccgcctcatcgagaagcaggcccctgagaacaaggccttcgtgctgagcagtgtggatgagctggagcagcaaagggacgagatcgttagctacctctgtgaccttgcccctgaagcccctcctcctactctgcccccccacatggcacaagtcactgtgggcccggggctcttgggggtttcgaccctggggcccaagaggaactccatggttctggatgtggcgttcgtcctggaaggatcggacaaaattggtgaagccgacttcaacaggagcaaggagttcatggaggaggtgattcagcggatggatgtgggccaggacagcatccacgtcacggtgctgcagtactcctacatggtgaccgtggagtaccccttcagcgaggcacagtccaaaggggacatcctgcagcgggtgcgagagatccgctaccagggcggcaacaggaccaacactgggctggccctgcggtacctctctgaccacagcttcttggtcagccagggtgaccgggagcaggcgcccaacctggtctacatggtcaccggaaatcctgcctctgatgagatcaagaggctgcctggagacatccaggtggtgcccattggagtgggccctaatgccaacgtgcaggagctggagaggattggctggcccaatgcccctatcctcatccaggactttgagacgctcccccgagaggctcctgacctggtgctgcagaggtgctgctccggagaggggctgcagatccccaccctctcccctgcacctgactgcagccagcccctggacgtgatccttctcctggatggctcctccagtttcccagcttcttattttgatgaaatgaagagtttcgccaaggctttcatttcaaaagccaatatagggcctcgtctcactcaggtgtcagtgctgcagtatggaagcatcaccaccattgacgtgccatggaacgtggtcccggagaaagcccatttgctgagccttgtggacgtcatgcagcgggagggaggccccagccaaatcggggatgccttgggctttgctgtgcgatacttgacttcagaaatgcatggtgccaggccgggagcctcaaaggcggtggtcatcctggtcacggacgtctctgtggattcagtggatgcagcagctgatgccgccaggtccaacagagtgacagtgttccctattggaattggagatcgctacgatgcagcccagctacggatcttggcaggcccagcaggcgactccaacgtggtgaagctccagcgaatcgaagacctccctaccatggtcaccttgggcaattccttcctccacaaactgtgctctggatttgttaggatttgcatggatgaggatgggaatgagaagaggcccggggacgtctggaccttgccagaccagtgccacaccgtgacttgccagccagatggccagaccttgctgaagagtcatcgggtcaactgtgaccgggggctgaggccttcgtgccctaacagccagtcccctgttaaagtggaagagacctgtggctgccgctggacctgcccctgcgtgtgcacaggcagctccactcggcacatcgtgacctttgatgggcagaatttcaagctgactggcagctgttcttatgtcctatttcaaaacaaggagcaggacctggaggtgattctccataatggtgcctgcagccctggagcaaggcagggctgcatgaaatccatcgaggtgaagcacagtgccctctccgtcgagctgcacagtgacatggaggtgacggtgaatgggagactggtctctgttccttacgtgggtgggaacatggaagtcaacgtttatggtgccatcatgcatgaggtcagattcaatcaccttggtcacatcttcacattcactccacaaaacaatgagttccaactgcagctcagccccaagacttttgcttcaaagacgtatggtctgtgtgggatctgtgatgagaacggagccaatgacttcatgctgagggatggcacagtcaccacagactggaaaacacttgttcaggaatggactgtgcagcggccagggcagacgtgccagcccatcctggaggagcagtgtcttgtccccgacagctcccactgccaggtcctcctcttaccactgtttgctgaatgccacaaggtcctggctccagccacattctatgccatctgccagcaggacagttgccaccaggagcaagtgtgtgaggtgatcgcctcttatgcccacctctgtcggaccaacggggtctgcgttgactggaggacacctgatttctgtgctatgtcatgcccaccatctctggtctacaaccactgtgagcatggctgtccccggcactgtgatggcaacgtgagctcctgtggggaccatccctccgaaggctgtttctgccctccagataaagtcatgttggaaggcagctgtgtccctgaagaggcctgcactcagtgcattggtgaggatggagtccagcaccagttcctggaagcctgggtcccggaccaccagccctgtcagatctgcacatgcctcagcgggcggaaggtcaactgcacaacgcagccctgccccacggccaaagctcccacgtgtggcctgtgtgaagtagcccgcctccgccagaatgcagaccagtgctgccccgagtatgagtgtgtgtgtgacccagtgagctgtgacctgcccccagtgcctcactgtgaacgtggcctccagcccacactgaccaaccctggcgagtgcagacccaacttcacctgcgcctgcaggaaggaggagtgcaaaagagtgtccccaccctcctgccccccgcaccgtttgcccacccttcggaagacccagtgctgtgatgagtatgagtgtgcctgcaactgtgtcaactccacagtgagctgtccccttgggtacttggcctcaaccgccaccaatgactgtggctgtaccacaaccacctgccttcccgacaaggtgtgtgtccaccgaagcaccatctaccctgtgggccagttctgggaggagggctgcgatgtgtgcacctgcaccgacatggaggatgccgtgatgggcctccgcgtggcccagtgctcccagaagccctgtgaggacagctgtcggtcgggcttcacttacgttctgcatgaaggcgagtgctgtggaaggtgcctgccatctgcctgtgaggtggtgactggctcaccgcggggggactcccagtcttcctggaagagtgtcggctcccagtgggcctccccggagaacccctgcctcatcaatgagtgtgtccgagtgaaggaggaggtctttatacaacaaaggaacgtctcctgcccccagctggaggtccctgtctgcccctcgggctttcagctgagctgtaagacctcagcgtgctgcccaagctgtcgctgtgagcgcatggaggcctgcatgctcaatggcactgtcattgggcccgggaagactgtgatgatcgatgtgtgcacgacctgccgctgcatggtgcaggtgggggtcatctctggattcaagctggagtgcaggaagaccacctgcaacccctgccccctgggttacaaggaagaaaataacacaggtgaatgttgtgggagatgtttgcctacggcttgcaccattcagctaagaggaggacagatcatgacactgaagcgtgatgagacgctccaggatggctgtgatactcacttctgcaaggtcaatgagagaggagagtacttctgggagaagagggtcacaggctgcccaccctttgatgaacacaagtgtctggctgagggaggtaaaattatgaaaattccaggcacctgctgtgacacatgtgaggagcctgagtgcaacgacatcactgccaggctgcagtatgtcaaggtgggaagctgtaagtctgaagtagaggtggatatccactactgccagggcaaatgtgccagcaaagccatgtactccattgacatcaacgatgtgcaggaccagtgctcctgctgctctccgacacggacggagcccatgcaggtggccctgcactgcaccaatggctctgttgtgtaccatgaggttctcaatgccatggagtgcaaatgctcccccaggaagtgcagcaagtgaggctgctgcagctgcatgggtgcctgctgctgcctgccttggcctgatggccaggccagagtgctgccagtcctctgcatgttctgctcttgtgcccttctgagcccacaataaaggctgagctcttatcttgctgcatgttctgctcttgtgcccttctgagcccacaat

SEQ ID NO: 8 - protein, 2813 aa, Human
MIPARFAGVLLALALILPGTLCAEGTRGRSSTARCSLFGSDFVNTFDGSMYSFAGYCSYLLAGGCQKRSFSIIGDFQNGKRVSLSVYLGEFFDIHLFVNGTVTQGDQRVSMPYASKGLYLETEAGYYKLSGEAYGFVARIDGSGNFQVLLSDRYFNKTCGLCGNFNIFAEDDFMTQEGTLTSDPYDFANSWALSSGEQWCERASPPSSSCNISSGEMQKGLWEQCQLLKSTSVFARCHPLVDPEPFVALCEKTLCECAGGLECACPALLEYARTCAQEGMVLYGWTDHSACSPVCPAGMEYRQCVSPCARTCQSLHINEMCQERCVDGCSCPEGQLLDEGLCVESTECPCVHSGKRYPPGTSLSRDCNTCICRNSQWICSNEECPGECLVTGQSHFKSFDNRYFTFSGICQYLLARDCQDHSFSIVIETVQCADDRDAVCTRSVTVRLPGLHNSLVKLKHGAGVAMDGQDIQLPLLKGDLRIQHTVTASVRLSYGEDLQMDWDGRGRLLVKLSPVYAGKTCGLCGNYNGNQGDDFLTPSGLAEPRVEDFGNAWKLHGDCQDLQKQHSDPCALNPRMTRFSEEACAVLTSPTFEACHRAVSPLPYLRNCRYDVCSCSDGRECLCGALASYAAACAGRGVRVAWREPGRCELNCPKGQVYLQCGTPCNLTCRSLSYPDEECNEACLEGCFCPPGLYMDERGDCVPKAQCPCYYDGEIFQPEDIFSDHHTMCYCEDGFMHCTMSGVPGSLLPDAVLSSPLSHRSKRSLSCRPPMVKLVCPADNLRAEGLECTKTCQNYDLECMSMGCVSGCLCPPGMVRHENRCVALERCPCFHQGKEYAPGETVKIGCNTCVCRDRKWNCTDHVCDATCSTIGMAHYLTFDGLKYLFPGECQYVLVQDYCGSNPGTFRILVGNKGCSHPSVKCKKRVTILVEGGEIELFDGEVNVKRPMKDETHFEVVESGRYIILLLGKALSVVWDRHLSISVVLKQTYQEKVCGLCGNFDGIQNNDLTSSNLQVEEDPVDFGNSWKVSSQCADTRKVPLDSSPATCHNNIMKQTMVDSSCRILTSDVFQDCNKLVDPEPYLDVCIYDTCSCESIGDCACFCDTIAAYAHVCAQHGKVVTWRTATLCPQSCEERNLRENGYECEWRYNSCAPACQVTCQHPEPLACPVQCVEGCHAHCPPGKILDELLQTCVDPEDCPVCEVAGRRFASGKKVTLNPSDPEHCQICHCDVVNLTCEACQEPGGLVVPPTDAPVSPTTLYVEDISEPPLHDFYCSRLLDLVFLLDGSSRLSEAEFEVLKAFVVDMMERLRISQKWVRVAVVEYHDGSHAYIGLKDRKRPSELRRIASQVKYAGSQVASTSEVLKYTLFQIFSKIDRPEASRIALLLMASQEPQRMSRNFVRYVQGLKKKKVIVIPVGIGPHANLKQIRLIEKQAPENKAFVLSSVDELEQQRDEIVSYLCDLAPEAPPPTLPPHMAQVTVGPGLLGVSTLGPKRNSMVLDVAFVLEGSDKIGEADFNRSKEFMEEVIQRMDVGQDSIHVTVLQYSYMVTVEYPFSEAQSKGDILQRVREIRYQGGNRTNTGLALRYLSDHSFLVSQGDREQAPNLVYMVTGNPASDEIKRLPGDIQVVPIGVGPNANVQELERIGWPNAPILIQDFETLPREAPDLVLQRCCSGEGLQIPTLSPAPDCSQPLDVILLLDGSSSFPASYFDEMKSFAKAFISKANIGPRLTQVSVLQYGSITTIDVPWNVVPEKAHLLSLVDVMQREGGPSQIGDALGFAVRYLTSEMHGARPGASKAVVILVTDVSVDSVDAAADAARSNRVTVFPIGIGDRYDAAQLRILAGPAGDSNVVKLQRIEDLPTMVTLGNSFLHKLCSGFVRICMDEDGNEKRPGDVWTLPDQCHTVTCQPDGQTLLKSHRVNCDRGLRPSCPNSQSPVKVEETCGCRWTCPCVCTGSSTRHIVTFDGQNFKLTGSCSYVLFQNKEQDLEVILHNGACSPGARQGCMKSIEVKHSALSVELHSDMEVTVNGRLVSVPYVGGNMEVNVYGAIMHEVRFNHLGHIFTFTPQNNEFQLQLSPKTFASKTYGLCGICDENGANDFMLRDGTVTTDWKTLVQEWTVQRPGQTCQPILEEQCLVPDSSHCQVLLLPLFAECHKVLAPATFYAICQQDSCHQEQVCEVIASYAHLCRTNGVCVDWRTPDFCAMSCPPSLVYNHCEHGCPRHCDGNVSSCGDHPSEGCFCPPDKVMLEGSCVPEEACTQCIGEDGVQHQFLEAWVPDHQPCQICTCLSGRKVNCTTQPCPTAKAPTCGLCEVARLRQNADQCCPEYECVCDPVSCDLPPVPHCERGLQPTLTNPGECRPNFTCACRKEECKRVSPPSCPPHRLPTLRKTQCCDEYECACNCVNSTVSCPLGYLASTATNDCGCTTTTCLPDKVCVHRSTIYPVGQFWEEGCDVCTCTDMEDAVMGLRVAQCSQKPCEDSCRSGFTYVLHEGECCGRCLPSACEVVTGSPRGDSQSSWKSVGSQWASPENPCLINECVRVKEEVFIQQRNVSCPQLEVPVCPSGFQLSCKTSACCPSCRCERMEACMLNGTVIGPGKTVMIDVCTTCRCMVQVGVISGFKLECRKTTCNPCPLGYKEENNTGECCGRCLPTACTIQLRGGQIMTLKRDETLQDGCDTHFCKVNERGEYFWEKRVTGCPPFDEHKCLAEGGKIMKIPGTCCDTCEEPECNDITARLQYVKVGSCKSEVEVDIHYCQGKCASKAMYSIDINDVQDQCSCCSPTRTEPMQVALHCTNGSVVYHEVLNAMECKCSPRKCSK

SEQ ID NO: 9 - DNA, 3345 bp, Human
aaccacgatgtctttggcacagcctctcatctgtcagatgggagcggggaccccggagagggagtcagccgaggtcctggcattccttgtgaacccccgtctgtgggtttctggtccagtgtcccttctccagattagatggcttaggcctcctctaagggggtgggcgtgcacatccggagagctgtctggtgtgcaggactgggctgcaggttaccctgaactgcaaccatcttagagcaaggcccagcttgcagcaggaggagctgcaggccgcccaccctagccacggcccctgccctggcaggaagcttccaagagtaaacactgcctaatcgtcccgcccagtagtgagcaggcctgtcccattccatactgaccagattcccagtcaccaaggccccctctcactccgctccactcctcgggctggctctcctgaggatgcaccagcgtcacccccgggcaagatgccctcccctctgtgtggccggaatccttgcctgtggctttctcctgggctgctggggaccctcccatttccagcagagttgtcttcaggctttggagccacaggccgtgtcttcttacttgagccctggtgctcccttaaaaggccgccctccttcccctggcttccagaggcagaggcagaggcagaggcgggctgcaggcggcatcctacacctggagctgctggtggccgtgggccccgatgtcttccaggctcaccaggaggacacagagcgctatgtgctcaccaacctcaacatcggggcagaactgcttcgggacccgtccctgggggctcagtttcgggtgcacctggtgaagatggtcattctgacagagcctgagggtgccccaaatatcacagccaacctcacctcgtccctgctgagcgtctgtgggtggagccagaccatcaaccctgaggacgacacggatcctggccatgctgacctggtcctctatatcactaggtttgacctggagttgcctgatggtaaccggcaggtgcggggcgtcacccagctgggcggtgcctgctccccaacctggagctgcctcattaccgaggacactggcttcgacctgggagtcaccattgcccatgagattgggcacagcttcggcctggagcacgacggcgcgcccggcagcggctgcggccccagcggacacgtgatggcttcggacggcgccgcgccccgcgccggcctcgcctggtccccctgcagccgccggcagctgctgagcctgctcagacccgtccctccgtcgccgctccctctgctggccacccacctctgcgccggcaggagccttagtcttggtcccagccaagagccggctcctggtggggggcgcgggccgagaactcctgttcccactcacaaaaggccacgcttccaaacgcttccatcctcgtgcccactcctccgtcccgcctcctcccggtgtacaccccgggactgagccgggcctgagccgggccttgtcgcagcgcaggacgggcgcgctgcgtgtgggacccgccgcggcctcaacccgggtccgcggggcacccgccggatgcgcagcctggcctctactacagcgccaacgagcagtgccgcgtggccttcggccccaaggctgtcgcctgcaccttcgccagggagcacctggtgagtctgccggcggtggcctgggattggctgtgaggtccctccgcatcacccagctcacgtcccccaaaacgtgcatggatatgtgccaggccctctcctgccacacagacccgctggaccaaagcagctgcagccgcctcctcgttcctctcctggatgggacagaatgtggcgtggagaagtggtgctccaagggtcgctgccgctccctggtggagctgacccccatagcagcagtgcatgggcgctggtctagctggggtccccgaagtccttgctcccgctcctgcggaggaggtgtggtcaccaggaggcggcagtgcaacaaccccagacctgcctttggggggcgtgcatgtgttggtgctgacctccaggccgagatgtgcaacactcaggcctgcgagaagacccagctggagttcatgtcgcaacagtgcgccaggaccgacggccagccgctgcgctcctcccctggcggcgcctccttctaccactggggtgctgctgtaccacacagccaaggggatgctctgtgcagacacatgtgccgggccattggcgagagcttcatcatgaagcgtggagacagcttcctcgatgggacccggtgtatgccaagtggcccccgggaggacgggaccctgagcctgtgtgtgtcgggcagctgcaggacatttggctgtgatggtaggatggactcccagcaggtatgggacaggtgccaggtgtgtggtggggacaacagcacgtgcagcccacggaagggctctttcacagctggcagagcgagagaatatgtcacatttctgacagttacccccaacctgaccagtgtctacattgccaaccacaggcctctcttcacacacttggcggtgaggatcggagggcgctatgtcgtggctgggaagatgagcatctcccctaacaccacctacccctccctcctggaggatggtcgtgtcgagtacagagtggccctcaccgaggaccggctgccccgcctggaggagatccgcatctggggacccctccaggaagatgctgacatccaggtgggaggtgtcagagcccagctcatgcacatcagctggtggagcaggcctggccttggagaacgagacctgtgtgccaggggcagatggcctggaggctccagtgactgaggggcctggctccgtagatgagaagctgcctgcccctgagccctgtgtcgggatgtcatgtcctccaggctggggccatctggatgccacctctgcaggggagaaggctccctccccatggggcagcatcaggacgggggctcaagctgcacacgtgtggacccctgtgcagggtcgtgctccgtctcctgcgggcgaggtctgatggagctgcgtttcctgtgcatggactctgccctcagggtgcctgtccaggaagagctgtgtggcctggcaagcaagcctgggagccggcgggaggtctgccaggctgtcccgtgccctgctcggtggcagtacaagctggcggcctgcagcgtgagctgtgggagaggggtcgtgcggaggatcctgtattgtgcccgggcccatggggaggacgatggtgaggagatcctgttggacacccagtgccaggggctgcctcgcccggaaccccaggaggcctgcagcctggagccctg

SEQ ID NO: 10 - DNA, 4782 bp, Human
aaccacgatgtctttggcacagcctctcatctgtcagatgggagcggggaccccggagagggagtcagccgaggtcctggcattccttgtgaacccccgtctgtgggtttctggtccagtgtcccttctccagattagatggcttaggcctcctctaagggggtgggcgtgcacatccggagagctgtctggtgtgcaggactgggctgcaggttaccctgaactgcaaccatcttagagcaaggcccagcttgcagcaggaggagctgcaggccgcccaccctagccacggcccctgccctggcaggaagcttccaagagtaaacactgcctaatcgtcccgcccagtagtgagcaggcctgtcccattccatactgaccagattcccagtcaccaaggccccctctcactccgctccactcctcgggctggctctcctgaggatgcaccagcgtcacccccgggcaagatgccctcccctctgtgtggccggaatccttgcctgtggctttctcctgggctgctggggaccctcccatttccagcagagttgtcttcaggctttggagccacaggccgtgtcttcttacttgagccctggtgctcccttaaaaggccgccctccttcccctggcttccagaggcagaggcagaggcagaggcgggctgcaggcggcatcctacacctggagctgctggtggccgtgggccccgatgtcttccaggctcaccaggaggacacagagcgctatgtgctcaccaacctcaacatcggggcagaactgcttcgggacccgtccctgggggctcagtttcgggtgcacctggtgaagatggtcattctgacagagcctgagggtgccccaaatatcacagccaacctcacctcgtccctgctgagcgtctgtgggtggagccagaccatcaaccctgaggacgacacggatcctggccatgctgacctggtcctctatatcactaggtttgacctggagttgcctgatggtaaccggcaggtgcggggcgtcacccagctgggcggtgcctgctccccaacctggagctgcctcattaccgaggacactggcttcgacctgggagtcaccattgcccatgagattgggcacagcttcggcctggagcacgacggcgcgcccggcagcggctgcggccccagcggacacgtgatggcttcggacggcgccgcgccccgcgccggcctcgcctggtccccctgcagccgccggcagctgctgagcctgctcagcgcaggacgggcgcgctgcgtgtgggacccgccgcggcctcaacccgggtccgcggggcacccgccggatgcgcagcctggcctctactacagcgccaacgagcagtgccgcgtggccttcggccccaaggctgtcgcctgcaccttcgccagggagcacctggatatgtgccaggccctctcctgccacacagacccgctggaccaaagcagctgcagccgcctcctcgttcctctcctggatgggacagaatgtggcgtggagaagtggtgctccaagggtcgctgccgctccctggtggagctgacccccatagcagcagtgcatgggcgctggtctagctggggtccccgaagtccttgctcccgctcctgcggaggaggtgtggtcaccaggaggcggcagtgcaacaaccccagacctgcctttggggggcgtgcatgtgttggtgctgacctccaggccgagatgtgcaacactcaggcctgcgagaagacccagctggagttcatgtcgcaacagtgcgccaggaccgacggccagccgctgcgctcctcccctggcggcgcctccttctaccactggggtgctgctgtaccacacagccaaggggatgctctgtgcagacacatgtgccgggccattggcgagagcttcatcatgaagcgtggagacagcttcctcgatgggacccggtgtatgccaagtggcccccgggaggacgggaccctgagcctgtgtgtgtcgggcagctgcaggacatttggctgtgatggtaggatggactcccagcaggtatgggacaggtgccaggtgtgtggtggggacaacagcacgtgcagcccacggaagggctctttcacagctggcagagcgagagaatatgtcacatttctgacagttacccccaacctgaccagtgtctacattgccaaccacaggcctctcttcacacacttggcggtgaggatcggagggcgctatgtcgtggctgggaagatgagcatctcccctaacaccacctacccctccctcctggaggatggtcgtgtcgagtacagagtggccctcaccgaggaccggctgccccgcctggaggagatccgcatctggggacccctccaggaagatgctgacatccaggtttacaggcggtatggcgaggagtatggcaacctcacccgcccagacatcaccttcacctacttccagcctaagccacggcaggcctgggtgtgggccgctgtgcgtgggccctgctcggtgagctgtggggcagggctgcgctgggtaaactacagctgcctggaccaggccaggaaggagttggtggagactgtccagtgccaagggagccagcagccaccagcgtggccagaggcctgcgtgctcgaaccctgccctccctactgggcggtgggagacttcggcccatgcagcgcctcctgtgggggcggcctgcgggagcggccagtgcgctgcgtggaggcccagggcagcctcctgaagacattgcccccagcccggtgcagagcaggggcccagcagccagctgtggcgctggaaacctgcaacccccagccctgccctgccaggtgggaggtgtcagagcccagctcatgcacatcagctggtggagcaggcctggccttggagaacgagacctgtgtgccaggggcagatggcctggaggctccagtgactgaggggcctggctccgtagatgagaagctgcctgcccctgagccctgtgtcgggatgtcatgtcctccaggctggggccatctggatgccacctctgcaggggagaaggctccctccccatggggcagcatcaggacgggggctcaagctgcacacgtgtggacccctgtggcagggtcgtgctccgtctcctgcgggcgaggtctgatggagctgcgtttcctgtgcatggactctgccctcagggtgcctgtccaggaagagctgtgtggcctggcaagcaagcctgggagccggcgggaggtctgccaggctgtcccgtgccctgctcggtggcagtacaagctggcggcctgcagcgtgagctgtgggagaggggtcgtgcggaggatcctgtattgtgcccgggcccatggggaggacgatggtgaggagatcctgttggacacccagtgccaggggctgcctcgcccggaaccccaggaggcctgcagcctggagccctgcccacctaggtggaaagtcatgtcccttggcccatgttcggccagctgtggccttggcactgctagacgctcggtggcctgtgtgcagctcgaccaaggccaggacgtggaggtggacgaggcggcctgtgcggcgctggtgcggcccgaggccagtgtcccctgtctcattgccgactgcacctaccgctggcatgttggcacctggatggagtgctctgtttcctgtggggatggcatccagcgccggcgtgacacctgcctcggaccccaggcccaggcgcctgtgccagctgatttctgccagcacttgcccaagccggtgactgtgcgtggctgctgggctgggccctgtgtgggacagggtgcctgtggcaggcagcaccttgagccaacaggaaccattgacatgcgaggcccagggcaggcagactgtgcagtggccattgggcggcccctcggggaggtggtgaccctccgcgtccttgagagttctctcaactgcagtgcgggggacatgttgctgctttggggccggctcacctggaggaagatgtgcaggaagctgttggacatgactttcagctccaagaccaacacgctggtggtgaggcagcgctgcgggcggccaggaggtggggtgctgctgcggtatgggagccagcttgctcctgaaaccttctacagagaatgtgacatgcagctctttgggccctggggtgaaatcgtgagcccctcgctgagtccagccacgagtaatgcagggggctgccggctcttcattaatgtggctccgcacgcacggattgccatccatgccctggccaccaacatgggcgctgggaccgagggagccaatgccagctacatcttgatccgggacacccacagcttgaggaccacagcgttccatgggcagcaggtgctctactgggagtcagagagcagccaggctgagatggagttcagcgagggcttcctgaaggctcaggccagcctgcggggccagtactggaccctccaatcatgggtaccggagatgcaggaccctcagtcctggaagggaaaggaaggaacctgagggtcattgaacatttgttccgtgtctggccagccctggagggttgacccctggtctcagtgctttccaattcgaactttttccaatcttaggtatctactttagagtcttctccaatgtccaaaaggctagggggttggaggtggggactctggaaaagcagcccccatttcctcgggtaccaataaataaaacatgcaggctgaaaaaaaaaaaaaaaaa

SEQ ID NO: 11 - DNA, 4689 bp, Human
aaccacgatgtctttggcacagcctctcatctgtcagatgggagcggggaccccggagagggagtcagccgaggtcctggcattccttgtgaacccccgtctgtgggtttctggtccagtgtcccttctccagattagatggcttaggcctcctctaagggggtgggcgtgcacatccggagagctgtctggtgtgcaggactgggctgcaggttaccctgaactgcaaccatcttagagcaaggcccagcttgcagcaggaggagctgcaggccgcccaccctagccacggcccctgccctggcaggaagcttccaagagtaaacactgcctaatcgtcccgcccagtagtgagcaggcctgtcccattccatactgaccagattcccagtcaccaaggccccctctcactccgctccactcctcgggctggctctcctgaggatgcaccagcgtcacccccgggcaagatgccctcccctctgtgtggccggaatccttgcctgtggctttctcctgggctgctggggaccctcccatttccagcagagttgtcttcaggctttggagccacaggccgtgtcttcttacttgagccctggtgctcccttaaaaggccgccctccttcccctggcttccagaggcagaggcagaggcagaggcgggctgcaggcggcatcctacacctggagctgctggtggccgtgggccccgatgtcttccaggctcaccaggaggacacagagcgctatgtgctcaccaacctcaacatcggggcagaactgcttcgggacccgtccctgggggctcagtttcgggtgcacctggtgaagatggtcattctgacagagcctgagggtgccccaaatatcacagccaacctcacctcgtccctgctgagcgtctgtgggtggagccagaccatcaaccctgaggacgacacggatcctggccatgctgacctggtcctctatatcactaggtttgacctggagttgcctgatggtaaccggcaggtgcggggcgtcacccagctgggcggtgcctgctccccaacctggagctgcctcattaccgaggacactggcttcgacctgggagtcaccattgcccatgagattgggcacagcttcggcctggagcacgacggcgcgcccggcagcggctgcggccccagcggacacgtgatggcttcggacggcgccgcgccccgcgccggcctcgcctggtccccctgcagccgccggcagctgctgagcctgctcagcgccaacgagcagtgccgcgtggccttcggccccaaggctgtcgcctgcaccttcgccagggagcacctggatatgtgccaggccctctcctgccacacagacccgctggaccaaagcagctgcagccgcctcctcgttcctctcctggatgggacagaatgtggcgtggagaagtggtgctccaagggtcgctgccgctccctggtggagctgacccccatagcagcagtgcatgggcgctggtctagctggggtccccgaagtccttgctcccgctcctgcggaggaggtgtggtcaccaggaggcggcagtgcaacaaccccagacctgcctttggggggcgtgcatgtgttggtgctgacctccaggccgagatgtgcaacactcaggcctgcgagaagacccagctggagttcatgtcgcaacagtgcgccaggaccgacggccagccgctgcgctcctcccctggcggcgcctccttctaccactggggtgctgctgtaccacacagccaaggggatgctctgtgcagacacatgtgccgggccattggcgagagcttcatcatgaagcgtggagacagcttcctcgatgggacccggtgtatgccaagtggcccccgggaggacgggaccctgagcctgtgtgtgtcgggcagctgcaggacatttggctgtgatggtaggatggactcccagcaggtatgggacaggtgccaggtgtgtggtggggacaacagcacgtgcagcccacggaagggctctttcacagctggcagagcgagagaatatgtcacatttctgacagttacccccaacctgaccagtgtctacattgccaaccacaggcctctcttcacacacttggcggtgaggatcggagggcgctatgtcgtggctgggaagatgagcatctcccctaacaccacctacccctccctcctggaggatggtcgtgtcgagtacagagtggccctcaccgaggaccggctgccccgcctggaggagatccgcatctggggacccctccaggaagatgctgacatccaggtttacaggcggtatggcgaggagtatggcaacctcacccgcccagacatcaccttcacctacttccagcctaagccacggcaggcctgggtgtgggccgctgtgcgtgggccctgctcggtgagctgtggggcagggctgcgctgggtaaactacagctgcctggaccaggccaggaaggagttggtggagactgtccagtgccaagggagccagcagccaccagcgtggccagaggcctgcgtgctcgaaccctgccctccctactgggcggtgggagacttcggcccatgcagcgcctcctgtgggggcggcctgcgggagcggccagtgcgctgcgtggaggcccagggcagcctcctgaagacattgcccccagcccggtgcagagcaggggcccagcagccagctgtggcgctggaaacctgcaacccccagccctgccctgccaggtgggaggtgtcagagcccagctcatgcacatcagctggtggagcaggcctggccttggagaacgagacctgtgtgccaggggcagatggcctggaggctccagtgactgaggggcctggctccgtagatgagaagctgcctgcccctgagccctgtgtcgggatgtcatgtcctccaggctggggccatctggatgccacctctgcaggggagaaggctccctccccatggggcagcatcaggacgggggctcaagctgcacacgtgtggacccctgcggcagggtcgtgctccgtctcctgcgggcgaggtctgatggagctgcgtttcctgtgcatggactctgccctcagggtgcctgtccaggaagagctgtgtggcctggcaagcaagcctgggagccggcgggaggtctgccaggctgtcccgtgccctgctcggtggcagtacaagctggcggcctgcagcgtgagctgtgggagaggggtcgtgcggaggatcctgtattgtgcccgggcccatggggaggacgatggtgaggagatcctgttggacacccagtgccaggggctgcctcgcccggaaccccaggaggcctgcagcctggagccctgcccacctaggtggaaagtcatgtcccttggcccatgttcggccagctgtggccttggcactgctagacgctcggtggcctgtgtgcagctcgaccaaggccaggacgtggaggtggacgaggcggcctgtgcggcgctggtgcggcccgaggccagtgtcccctgtctcattgccgactgcacctaccgctggcatgttggcacctggatggagtgctctgtttcctgtggggatggcatccagcgccggcgtgacacctgcctcggaccccaggcccaggcgcctgtgccagctgatttctgccagcacttgcccaagccggtgactgtgcgtggctgctgggctgggccctgtgtgggacagggtgcctgtggcaggcagcaccttgagccaacaggaaccattgacatgcgaggcccagggcaggcagactgtgcagtggccattgggcggcccctcggggaggtggtgaccctccgcgtccttgagagttctctcaactgcagtgcgggggacatgttgctgctttggggccggctcacctggaggaagatgtgcaggaagctgttggacatgactttcagctccaagaccaacacgctggtggtgaggcagcgctgcgggcggccaggaggtggggtgctgctgcggtatgggagccagcttgctcctgaaaccttctacagagaatgtgacatgcagctctttgggccctggggtgaaatcgtgagcccctcgctgagtccagccacgagtaatgcagggggctgccggctcttcattaatgtggctccgcacgcacggattgccatccatgccctggccaccaacatgggcgctgggaccgagggagccaatgccagctacatcttgatccgggacacccacagcttgaggaccacagcgttccatgggcagcaggtgctctactgggagtcagagagcagccaggctgagatggagttcagcgagggcttcctgaaggctcaggccagcctgcggggccagtactggaccctccaatcatgggtaccggagatgcaggaccctcagtcctggaagggaaaggaaggaacctgagggtcattgaacatttgttccgtgtctggccagccctggagggttgacccctggtctcagtgctttccaattcgaactttttccaatcttaggtatctactttagagtcttctccaatgtccaaaaggctagggggttggaggtggggactctggaaaagcagcccccatttcctcgggtaccaataaataaaacatgcaggctgaaaaaaaaaaaaaaaaa

SEQ ID NO: 12 - DNA, 4950 bp, Human
aaccacgatgtctttggcacagcctctcatctgtcagatgggagcggggaccccggagagggagtcagccgaggtcctggcattccttgtgaacccccgtctgtgggtttctggtccagtgtcccttctccagattagatggcttaggcctcctctaagggggtgggcgtgcacatccggagagctgtctggtgtgcaggactgggctgcaggttaccctgaactgcaaccatcttagagcaaggcccagcttgcagcaggaggagctgcaggccgcccaccctagccacggcccctgccctggcaggaagcttccaagagtaaacactgcctaatcgtcccgcccagtagtgagcaggcctgtcccattccatactgaccagattcccagtcaccaaggccccctctcactccgctccactcctcgggctggctctcctgaggatgcaccagcgtcacccccgggcaagatgccctcccctctgtgtggccggaatccttgcctgtggctttctcctgggctgctggggaccctcccatttccagcagagttgtcttcaggctttggagccacaggccgtgtcttcttacttgagccctggtgctcccttaaaaggccgccctccttcccctggcttccagaggcagaggcagaggcagaggcgggctgcaggcggcatcctacacctggagctgctggtggccgtgggccccgatgtcttccaggctcaccaggaggacacagagcgctatgtgctcaccaacctcaacatcggggcagaactgcttcgggacccgtccctgggggctcagtttcgggtgcacctggtgaagatggtcattctgacagagcctgagggtgctccaaatatcacagccaacctcacctcgtccctgctgagcgtctgtgggtggagccagaccatcaaccctgaggacgacacggatcctggccatgctgacctggtcctctatatcactaggtttgacctggagttgcctgatggtaaccggcaggtgcggggcgtcacccagctgggcggtgcctgctccccaacctggagctgcctcattaccgaggacactggcttcgacctgggagtcaccattgcccatgagattgggcacagcttcggcctggagcacgacggcgcgcccggcagcggctgcggccccagcggacacgtgatggcttcggacggcgccgcgccccgcgccggcctcgcctggtccccctgcagccgccggcagctgctgagcctgctcagcgcaggacgggcgcgctgcgtgtgggacccgccgcggcctcaacccgggtccgcggggcacccgccggatgcgcagcctggcctctactacagcgccaacgagcagtgccgcgtggccttcggccccaaggctgtcgcctgcaccttcgccagggagcacctggatatgtgccaggccctctcctgccacacagacccgctggaccaaagcagctgcagccgcctcctcgttcctctcctggatgggacagaatgtggcgtggagaagtggtgctccaagggtcgctgccgctccctggtggagctgacccccatagcagcagtgcatgggcgctggtctagctggggtccccgaagtccttgctcccgctcctgcggaggaggtgtggtcaccaggaggcggcagtgcaacaaccccagacctgcctttggggggcgtgcatgtgttggtgctgacctccaggccgagatgtgcaacactcaggcctgcgagaagacccagctggagttcatgtcgcaacagtgcgccaggaccgacggccagccgctgcgctcctcccctggcggcgcctccttctaccactggggtgctgctgtaccacacagccaaggggatgctctgtgcagacacatgtgccgggccattggcgagagcttcatcatgaagcgtggagacagcttcctcgatgggacccggtgtatgccaagtggcccccgggaggacgggaccctgagcctgtgtgtgtcgggcagctgcaggacatttggctgtgatggtaggatggactcccagcaggtatgggacaggtgccaggtgtgtggtggggacaacagcacgtgcagcccacggaagggctctttcacagctggcagagcgagagaatatgtcacatttctgacagttacccccaacctgaccagtgtctacattgccaaccacaggcctctcttcacacacttggcggtgaggatcggagggcgctatgtcgtggctgggaagatgagcatctcccctaacaccacctacccctccctcctggaggatggtcgtgtcgagtacagagtggccctcaccgaggaccggctgccccgcctggaggagatccgcatctggggacccctccaggaagatgctgacatccaggtttacaggcggtatggcgaggagtatggcaacctcacccgcccagacatcaccttcacctacttccagcctaagccacggcaggcctgggtgtgggccgctgtgcgtgggccctgctcggtgagctgtggggcagggctgcgctgggtaaactacagctgcctggaccaggccaggaaggagttggtggagactgtccagtgccaagggagccagcagccaccagcgtggccagaggcctgcgtgctcgaaccctgccctccctactgggcggtgggagacttcggcccatgcagcgcctcctgtgggggcggcctgcgggagcggccagtgcgctgcgtggaggcccagggcagcctcctgaagacattgcccccagcccggtgcagagcaggggcccagcagccagctgtggcgctggaaacctgcaacccccagccctgccctgccaggtgggaggtgtcagagcccagctcatgcacatcagctggtggagcaggcctggccttggagaacgagacctgtgtgccaggggcagatggcctggaggctccagtgactgaggggcctggctccgtagatgagaagctgcctgcccctgagccctgtgtcgggatgtcatgtcctccaggctggggccatctggatgccacctctgcaggggagaaggctccctccccatggggcagcatcaggacgggggctcaagctgcacacgtgtggacccctgcggcagggtcgtgctccgtctcctgcgggcgaggtctgatggagctgcgtttcctgtgcatggactctgccctcagggtgcctgtccaggaagagctgtgtggcctggcaagcaagcctgggagccggcgggaggtctgccaggctgtcccgtgccctgctcggtggcagtacaagctggcggcctgcagcgtgagctgtgggagaggggtcgtgcggaggatcctgtattgtgcccgggcccatggggaggacgatggtgaggagatcctgttggacacccagtgccaggggctgcctcgcccggaaccccaggaggcctgcagcctggagccctgcccacctaggtggaaagtcatgtcccttggcccatgttcggccagctgtggccttggcactgctagacgctcggtggcctgtgtgcagctcgaccaaggccaggacgtggaggtggacgaggcggcctgtgcggcgctggtgcggcccgaggccagtgtcccctgtctcattgccgactgcacctaccgctggcatgttggcacctggatggagtgctctgtttcctgtggggatggcatccagcgccggcgtgacacctgcctcggaccccaggcccaggcgcctgtgccagctgatttctgccagcacttgcccaagccggtgactgtgcgtggctgctgggctgggccctgtgtgggacagggtacgcccagcctggtgccccacgaagaagccgctgctccaggacggaccacagccacccctgctggtgcctccctggagtggtcccaggcccggggcctgctcttctccccggctccccagcctcggcggctcctgcccgggccccaggaaaactcagtgcagtccagtgcctgtggcaggcagcaccttgagccaacaggaaccattgacatgcgaggcccagggcaggcagactgtgcagtggccattgggcggcccctcggggaggtggtgaccctccgcgtccttgagagttctctcaactgcagtgcgggggacatgttgctgctttggggccggctcacctggaggaagatgtgcaggaagctgttggacatgactttcagctccaagaccaacacgctggtggtgaggcagcgctgcgggcggccaggaggtggggtgctgctgcggtatgggagccagcttgctcctgaaaccttctacagagaatgtgacatgcagctctttgggccctggggtgaaatcgtgagcccctcgctgagtccagccacgagtaatgcagggggctgccggctcttcattaatgtggctccgcacgcacggattgccatccatgccctggccaccaacatgggcgctgggaccgagggagccaatgccagctacatcttgatccgggacacccacagcttgaggaccacagcgttccatgggcagcaggtgctctactgggagtcagagagcagccaggctgagatggagttcagcgagggcttcctgaaggctcaggccagcctgcggggccagtactggaccctccaatcatgggtaccggagatgcaggaccctcagtcctggaagggaaaggaaggaacctgagggtcattgaacatttgttccgtgtctggccagccctggagggttgacccctggtctcagtgctttccaattcgaactttttccaatcttaggtatctactttagagtcttctccaatgtccaaaaggctagggggttggaggtggggactctggaaaagcagcccccatttcctcgggtaccaataaataaaacatgcaggctgaaaaaaaaaaaaaaaaa

SEQ ID NO: 13 - protein, 12 aa, Artificial Sequence
  epitope tag
MRGSHHHHHHGS

SEQ ID NO: 14 - protein, 11 aa, Artificial Sequence
  epitope tag
ETARFQPGYRS

SEQ ID NO: 15 - protein, 10 aa, Artificial Sequence
  epitope tag
EQKLISEEDL

SEQ ID NO: 16 - protein, 8 aa, Artificial Sequence
  epitope tag
DYKDDDDK

SEQ ID NO: 17 - protein, 12 aa, Artificial Sequence
  epitope tag
GVSSTSSDFRDR

SEQ ID NO: 18 - protein, 9 aa, Artificial Sequence
  epitope tag
YPYDVPDYA

SEQ ID NO: 19 - DNA, 30 bp, Artificial Sequence
  Primer
aaggccggatccgggctcttgggggtttcg

SEQ ID NO: 20 - DNA, 30 bp, Artificial Sequence
  Primer
aaggccaagcttcctctgcagcaccaggtc

SEQ ID NO: 21 - DNA, 564 bp, Human
gggctcttgggggtttcgaccctggggcccaagaggaactccatggttctggatgtggcgttcgtcctggaaggatcggacaaaattggtgaagccgacttcaacaggagcaaggagttcatggaggaggtgattcagcggatggatgtgggccaggacagcatccacgtcacggtgctgcagtactcctacatggtgaccgtggagtaccccttcagcgaggcacagtccaaaggggacatcctgcagcgggtgcgagagatccgctaccagggcggcaacaggaccaacactgggctggccctgcggtacctctctgaccacagcttcttggtcagccagggtgaccgggagcaggcgcccaacctggtctacatggtcaccggaaatcctgcctctgatgagatcaagaggctgcctggagacatccaggtggtgcccattggagtgggccctaatgccaacgtgcaggagctggagaggattggctggcccaatgcccctatcctcatccaggactttgagacgctcccccgagaggctcctgacctggtgctgcagagg

SEQ ID NO: 22 - DNA, 30 bp, Artificial Sequence; identical to SEQ ID NO: 19
  Primer

SEQ ID NO: 23 - DNA, 31 bp, Artificial Sequence
  Primer
aaggccgtcgactcctctgcagcaccaggtc

(19) United States
(12) Patent Application Publication
(10) Pub. No.: US 2005/0186646 A1
(43) Pub. Date: Aug. 25, 2005

(54) Rapid assay to detect ADAMTS-13 activity

(76) Inventor: Miguel A. Cruz, Pearland, TX (US)

Correspondence Address: FULBRIGHT & JAWORSKI, LLP, 1301 MCKINNEY, SUITE 5100, HOUSTON, TX, 77010-3095, US

(21) Appl. No.: 11/043,611

(22) Filed: Jan. 26, 2005

Related U.S. Application Data
(60) Provisional application No. 60/539,308, filed on Jan. 26, 2004.

(51) Int. Cl. G01N 33/53; G01N 33/537; G01N 33/543

(52) U.S. Cl. 435/7.92

(57) ABSTRACT

The present invention is directed to an assay for ADAMTS-13 activity. The assay provides rapid analysis of ADAMTS-13 activity in a biological sample under non-denaturing conditions. Furthermore, the present invention is directed to a recombinant polypeptide with a functional ADAMTS-13 site that is useful in ADAMTS-13 activity assays.

This application claims priority to U.S. Provisional Application No. 60/539,308, which was filed Jan. 26, 2004, and which is hereby incorporated by reference it its entirety herein.

### TECHNICAL FIELD

The present invention is related to the fields of genetics, molecular biology, cell biology, and medicine. The invention is directed to an assay for the detection of ADAMTS-13 activity. The invention is also directed to a recombinant protein useful for the assay.

### BACKGROUND OF THE INVENTION

The metalloprotease ADAMTS-13 cleaves the ‘unusually large’ (UL) von Willebrand factor (VWF) multimers newly released from the endothelial cells under physiological flowing conditions. VWF multimers are cleaved at the Y1605-M1606 (SEQ ID NO:8) peptide bond within the VWF-A2 domain (Dent et al., 1990; Tsai et al., 1996; Furlan et al., 1996). The critical function of the ADAMTS-13 is underscored by clinical thrombotic manifestations caused by the deficiency of this metalloprotease in patients with thrombotic thrombocytopenic purpura (TTP). Deficiency of the enzyme activity in these patients results in the plasma accumulation of the ULVWF multimers, which bind spontaneously with circulating platelets, leading to platelet aggregation and thrombotic micro angiopathy (TM) (Moake et al., 1982; Moake et al., 1985; Moake et al., 1994).

Although deficiency of ADAMTS-13 activity is associated with TM and is often diagnosed as TTP, reduced enzyme activity has also been found in inflammation, pregnancy, liver disease (Mannucci et al., 2001), disseminated intravascular coagulation (DIC) (Loof et al., 2001), sepsis and heparin-induced thrombocytopenia (Bianchi et al., 2002), suggesting that clinical measurement of ADAMTS-13 activity may serve as a marker not only for TTP, but also a variety of other diseases.

Furlan et al. (1998) and Tsai et al. (1998) initially developed assays to measure ADAMTS-13 activity in plasma. Both methods use immunoblotting to observe either the disappearance of large VWF multimers or the appearance of 176 kDa and 140 kDa dimers. The dimeric fragments represent each side of the Y1605-M1606 cleavage site in the A2 domain of two disulfide-linked VWF monomers (Dent et al., 1990). Other methods to estimate ADAMTS-13 activity have also been reported. These include measurement of the collagen-binding activity of a cleaved VWF (Gerritsen et al., 1999), detection of cleaved VWF by antiVWF antibody in an ELISA He et al., 2001, and Obert et al., 1999), assays using endogenous VWF as substrate (Kirzek et al., 2001, and Aronson et al., 2001) and estimation of cleaved VWF multimeric size using ristocetin cofactor activity (Bohm et al., 2002). These assays are done under static conditions, and require long incubation times (up to 24 h), the addition of either Ba²⁺ or Ca²⁺ to activate the enzyme, and denaturing conditions (urea or guanidine). All these assays measure ADAMTS-13 activity in a non-physiological environment and they are also time consuming and costly.

In addition to the above static condition assays, there are two reports that used flow conditions to measure ADAMTS-13 activity. First, Shenkman et al. (2003) evaluated the ability of TTP plasma to increase the platelet deposition on a polystyrene surface from normal plasma under flow conditions using a cone and platelet analyzer (CPA) method. Second, Dong et al. (2002) described that ULVWF multimers, upon release, form extremely long and platelet-decorated string-like structures on the surface of endothelial cells under fluid shear stress. These ULVWF strings are rapidly cleaved by ADAMTS-13 in a kinetic that is 1000-fold faster than under static conditions. These results suggest that the process of ULVWF by ADAMTS-13 may occur on endothelial cells and provide a new way to measure ADAMTS-13 activity in a more physiologically relevant way. However, this flow-based assay is expensive and also time consuming in the preparation of endothelial cells. It is therefore highly desirable to develop a rapid and cost-effective clinical method to test ADAMTS-13 activity.

### BRIEF SUMMARY OF THE INVENTION

An embodiment of the invention is a method for detecting ADAMTS-13 activity in a biological sample comprising the steps of: collecting the biological sample; contacting the sample with a recombinant polypeptide comprising a functional ADAMTS-13 cleavage site; and measuring cleavage of said recombinant polypeptide, wherein the presence of cleaved recombinant polypeptide indicates ADAMTS-13 activity.

In a specific embodiment, measuring cleavage of the recombinant polypeptide comprises gel electrophoresis or immunoblotting. In a specific embodiment, measuring cleavage of the recombinant polypeptide comprises an ELISA.

In one embodiment of the invention, the recombinant polypeptide comprises the A2 domain of von Willebrand factor.

In other embodiment, the functional ADAMTS-13 cleavage site comprises the ADAMTS-13 cleavage site of von Willebrand factor.

In one embodiment, the recombinant polypeptide comprises SEQ ID NO: 1 The recombinant polypeptide comprises at least one epitope tag, in a specific embodiment. In a specific embodiment, the epitope tag is selected from the group consisting of histidine tag, tag-100, c-myc, FLAG, E2, HA and GST. In a further specific embodiment, the recombinant polypeptide further comprises a fluorescent tag.

In yet another embodiment of the invention, contacting takes place under non-denaturing conditions. In a specific embodiment, the contacting is for a period of time of less than about 2 hours. In a specific embodiment, the contacting is for a period of time of less than about 1 hours. In another specific embodiment, the contacting and measuring steps do not require the addition of ions.

An embodiment of the invention is a method of diagnosing a disease in a mammal associated with decreased ADAMTS-13 activity comprising the steps of: collecting a biological sample from the mammal; contacting the sample with a recombinant polypeptide comprising a functional ADAMTS-13 cleavage site; and measuring cleavage of said recombinant polypeptide, wherein a decreased level of cleaved recombinant polypeptide in the sample as compared to a control sample is diagnostic for the disease.

In one embodiment, the disease associated with decreased ADAMTS-13 activity is selected from the group consisting of TTP, DIC, sepsis, heparin-induced thrombocytopenia, thrombotic microangiopathy, cancer, and liver disease.

An embodiment of the invention is a method of identifying a mammal at risk of developing TTP disease comprising the steps of: collecting a biological sample from the mammal; contacting the sample with a recombinant polypeptide comprising a functional ADAMTS-13 cleavage site; and measuring cleavage of said recombinant polypeptide, wherein a decreased level of cleaved recombinant polypeptide in the sample as compared to a control sample indicates that the mammal is at risk of developing TTP.

An embodiment of the invention is a recombinant polypeptide comprising a functional ADAMTS-13 cleavage site and at least one epitope tag, wherein the at least one epitope tag is located on the N-terminal side or the C-terminal side of the ADAMTS-13 cleavage site. In a specific embodiment, the recombinant polypeptide further comprises a second epitope tag, wherein the second epitope tag is located on a side opposite the ADAMTS-13 cleavage site of the at least one epitope tag. In a specific embodiment, the epitope tag is selected from the group consisting of histidine tag, tag-100, c-myc, FLAG, E2, HA, and GST. In a specific embodiment, the second epitope tag is selected from the group consisting of histidine tag, tag-100, c-myc, FLAG, E2, and HA, and is different than the at least one epitope tag. In one embodiment of the invention, the epitope tag is selected from the group consisting of histidine tag, tag-100, c-myc, FLAG, E2, HA, and GST, the second epitope tag is selected from the group consisting of histidine tag, tag-100, c-myc, FLAG, E2, and HA, and the second epitope tag is different than the first epitope tag.

An embodiment of the invention is a host cell expressing the recombinant polypeptide described herein. An embodiment of the invention is a recombinant polypeptide comprising SEQ ID NO:1. In a specific embodiment, the recombinant polypeptide SEQ ID NO:1 further comprises at least one epitope tag.

An embodiment of the invention is a isolated nucleic acid encoding the recombinant polypeptide described herein.

An embodiment of the invention is a kit comprising the polypeptide described herein, an antibody specific for the first epitope tag, and an antibody specific for the second epitope tag. In specific embodiments, the kit also comprises a conjugated antibody, such as a peroxidase or FITC.

The foregoing has outlined rather broadly the features and technical advantages of the present invention in order that the detailed description of the invention that follows may be better understood. Additional features and advantages of the invention will be described hereinafter which form the subject of the claims of the invention. It should be appreciated that the conception and specific embodiment disclosed may be readily utilized as a basis for modifying or designing other structures for carrying out the same purposes of the present invention. It should also be realized that such equivalent constructions do not depart from the invention as set forth in the appended claims. The novel features which are believed to be characteristic of the invention, both as to its organization and method of operation, together with further objects and advantages will be better understood from the following description when considered in connection with the accompanying figures. It is to be expressly understood, however, that each of the figures is provided for the purpose of illustration and description only and is not intended as a definition of the limits of the present invention.

### BRIEF DESCRIPTION OF THE DRAWINGS

For a more complete understanding of the present invention, reference is now made to the following descriptions taken in conjunction with the accompanying drawing, in which:

FIG. 1A and FIG. 1B depict SDS-PAGE analysis of purified recombinant VWF-A2 protein with a single tag. FIG. 1A is a map of the recombinant VWF-A2 construct used for bacterial expression. FIG. 1B is a Coomassie blue stained SDS-PAGE verifying purity of the single-tagged bacterial VWF-A2 protein;

FIG. 2 shows analysis of ADAMTS-13 cleavage activity by Western blotting;

FIG. 3A and FIG. 3B show detection of recombinant VWF-A2 peptide by the monoclonal antibody Tag-100. FIG. 3A shows specific binding of intact A2 peptide. As the plasma concentration decreases the absorbance that represents bound intact A2 peptide increases. FIG. 3B shows the absorbance values corresponding to each plasma dilution were converted to percentage of ADAMTS-13 activity;

FIG. 4 shows activity of ADAMTS-13 in 39 normal controls and 16 samples from thrombocytopenic purpura (TTP) patient;

FIG. 5A and FIG. 5B show ADAMTS-13 specifically cleaves recombinant VWF-A2 peptide in the ELISA assay. FIG. 5A shows restoration of enzyme activity in congenital thrombocytopenic purpura (TTP) plasmas after they were mixed with normal plasma.

FIG. 5B shows three different dilutions of partially isolated ADAMTS-13 enzyme were used to demonstrate the ability of the enzyme to cleave recombinant VWF-A2 peptide; and

FIG. 6A and FIG. 6B show a comparison between the ELISA assay and two other methods. FIG. 6A shows a correlation between the ELISA assay and the quantitative immunoblotting method that uses recombinant VWF-A2 peptide as substrate. FIG. 6B shows a correlation between the ELISA and the long incubation period immunoblotting method that uses ‘unusually large’ von Willebrand factor (ULVWF) as substrate.

### DETAILED DESCRIPTION OF THE INVENTION

As used herein the specification, “a” or “an” may mean one or more. As used herein in the claim(s), when used in conjunction with the word “comprising”, the words “a” or “an” may mean one or more than one. As used herein “another” may mean at least a second or more. Still further, the terms “having”, “including”, “containing” and “comprising” are interchangeable and one of skill in the art is cognizant that these terms are open ended terms.

The term “biological sample” refers to a sample obtained from a mammal for the purpose of diagnosis, prognosis, or evaluation. In certain embodiments, such a sample may be obtained for the purpose of determining the outcome of an ongoing condition or the effect of a treatment regimen on a condition. Examples of biological samples are blood samples, serum samples, plasma samples, cerebrospinal fluid, tissue samples, and urine samples. In a preferred embodiment, the biological sample is a blood sample.

As used herein, the expressions “cell,” “cell line,” and “cell culture” are used interchangeably and all such designations include progeny. Thus, the words “transformants” and “transformed cells” include the primary subject cell and cultures derived therefrom without regard for the number of transfers. It is also understood that all progeny may not be precisely identical in DNA content, due to deliberate or inadvertent mutations. Mutant progeny that have the same function or biological activity as screened for in the originally transformed cell are included. Where distinct designations are intended, it will be clear from the context.

The term “domain” as used herein refers to a subsection of the polypeptide which possesses a unique structural and/or functional characteristic; typically, this characteristic is similar across diverse polypeptides. The subsection typically comprises contiguous amino acids, although it may also comprise amino acids which act in concert or which are in close proximity due to folding or other configurations. An example of a protein domain is the A2 domain of VWF.

“Epitope tags” are short peptide sequences that are easily recognized by tag-specific antibodies. In certain embodiments of the invention, nucleic acid sequences encoding the epitope tag are included with target DNA at the time of cloning to produce fusion proteins containing the epitope tag sequence. This allows anti-epitope tag antibodies to serve as detection reagents for any tag containing polypeptide produced by recombinant means. Anti-epitope tag antibodies are useful for identifying or purifying a recombinant protein containing a specific epitope tag. The anti-epitope tag antibody is usually functional in a variety of antibody-dependent experimental procedures. Expression vectors producing epitope tag fusion proteins are available for a variety of host expression systems including bacteria, yeast, insect and mammalian cells. Due to their small size, epitope tags do not affect the tagged protein's biochemical properties.

As used herein, an “ion” is any atom or atoms that bears one or more positive or negative electrical charges. Positively charged ions are “cations” and negatively charged ions are “anions.” In certain embodiments of the invention, the addition of ions is not required in order to detect ADAMTS-13 activity in a biological sample. In a specific embodiment, the addition of divalent cations is not required in order to detect ADAMTS-13 activity in a biological sample. In another specific embodiment, the addition of metal ions is not required in order to detect ADAMTS-13 activity in a biological sample. One with skill in the art realizes that biological samples normally contain some ions, and the phrase “addition of ions” does not refer to ions in amounts naturally present in the biological sample.

As used herein, a “mammal” is an appropriate subject for the method of the present invention. A mammal may be any member of the higher vertebrate class Mammalia, including humans; characterized by live birth, body hair, and mammary glands in the female that secrete milk for feeding the young. Additionally, mammals are characterized by their ability to maintain a constant body temperature despite changing climatic conditions. Examples of mammals are humans, cats, dogs, cows, mice, rats, and chimpanzees. Mammals may be referred to as “patients”.

Nucleic acid is “operably linked” when it is placed into a functional relationship with another nucleic acid sequence. For example, DNA for a presequence or secretory leader is operably linked to DNA for a polypeptide if it is expressed as a preprotein that participates in the secretion of the polypeptide; a promoter or enhancer is operably linked to a coding sequence if it affects the transcription of the sequence; or a ribosome binding site is operably linked to a coding sequence if it is positioned so as to facilitate translation. Generally, “operably linked” means that the DNA sequences being linked are contiguous and, in the case of a secretory leader, contiguous and in reading phase. However enhancers do not have to be contiguous. Linking is accomplished by ligation at convenient restriction sites. If such sites do not exist, then synthetic oligonucleotide adaptors or linkers are used in accord with conventional practice.

As used herein, “plasmids” are designated by a lower case p preceded and/or followed by capital letters and/or numbers. The starting plasmids herein are commercially available, are publicly available on an unrestricted basis, or can be constructed from such available plasmids in accord with published procedures. In addition, other equivalent plasmids are known in the art and will be apparent to the ordinary artisan.

“Recovery” or “isolation” of a given fragment of DNA from a restriction digest means separation of the digest on polyacrylamide or agarose gel by electrophoresis, identification of the fragment of interest by comparison of its mobility versus that of marker DNA fragments of known molecular weight, removal of the gel section containing the desired fragment, and separation of the gel from DNA. This procedure is known generally. For example, see Lawn et al., Nucleic Acids Res., 9:6103-6114 (1981), and Goeddel et al., Nucleic Acids Res. 8:4057 (1980).

The term “thrombotic thrombocytopenic purpura” or “TTP” refers to a disease characterized by intravascular destruction of erythrocytes and consumption of blood platelets resulting in anemia and thrombocytopenia. Diffuse platelet rich microthrombi are observed in multiple organs, with the major extravascular manifestations including fever, and variable degrees of neurologic and renal dysfunction. Purpura refers to the characteristic bleeding that occurs beneath the skin, or in mucus membranes, which produces bruises, or a red rash-like appearance.

The terms “variant” and “mutant” when used in reference to a nucleotide sequence refer to an nucleic acid sequence that differs by one or more nucleotides from another, usually related nucleotide acid sequence. A “variation” is a difference between two different nucleotide sequences; typically, one sequence is a reference sequence.

The term “wild-type” when made in reference to a gene refers to a gene that has the characteristics of a gene isolated from a naturally occurring source. The term “wild-type” when made in reference to a gene product refers to a gene product that has the characteristics of a gene product isolated from a naturally occurring source. The term “naturally-occurring” as applied to an object refers to the fact that an object can be found in nature. For example, a polypeptide or polynucleotide sequence that is present in an organism (including viruses) that can be isolated from a source in nature and which has not been intentionally modified by man in the laboratory is naturally-occurring. A wild-type gene is frequently that gene which is most frequently observed in a population and is thus arbitrarily designated the “normal” or “wild-type” form of the gene. In contrast, the term “modified” or “mutant” when made in reference to a gene or to a gene product refers, respectively, to a gene or to a gene product which displays modifications in sequence and/or functional properties (i.e., altered characteristics) when compared to the wild-type gene or gene product. It is noted that naturally-occurring mutants can be isolated; these are identified by the fact that they have altered characteristics when compared to the wild-type gene or gene product.

A. ADAMTS-13 and Functional ADAMTS-13 Cleavage Sites

The term “ADAMTS-13” refers to a protein encoded by ADAMTS-13, a gene responsible for familial TTP. ADAMTS-13 has been identified as a unique member of the metalloproteinase gene family, ADAM (a disintegrin and metalloproteinase), whose members are membrane-anchored proteases with diverse functions. ADAMTS family members are distinguished from ADAMs by the presence of one or more thrombospondin 1-like (TSP 1) domain(s) at the C-terminus and the absence of the EGF repeat, transmembrane domain and cytoplasmic tail typically observed in ADAM metalloproteinases. ADAMTS-13 possesses von Willebrand factor (VWF) cleaving protease activity. One with skill in the art realizes that ADAMTS-13 exists in polypeptide variants in the human population, and the term ADAMTS-13 encompasses all such variants. Examples of ADAMTS-13 proteins are SEQ ID NO:3 (GenBank Accession Number NP_{—}620594), SEQ ID NO:4 (GenBank Accession Number NP_{—}620595), SEQ ID NO:5 (GenBank Accession Number NP_{—}620596), and SEQ ID NO:6 (GenBank Accession Number NP_{—}620597).

As used herein, the term “functional ADAMTS-13 cleavage site” refers to any polypeptide sequence capable of being cleaved at a peptide bond by a wild-type ADAMTS-13 polypeptide. A physiological function of ADAMTS-13 is to cleave VWF (nucleotide sequence SEQ ID NO:7, GenBank Accession Number NM_{—}000552; protein sequence SEQ ID NO:8 GenBank Accession Number NP_{—}000543). In certain embodiments of the invention, the functional ADAMTS-13 cleavage site is able to be cleaved in non-denaturing conditions. In certain embodiments of the invention, the functional ADAMTS-13 cleavage site is able to be cleaved in physiological buffer saline. In another embodiment of the invention, the ADAMTS-13 is contacted with the recombinant protein containing the functional ADAMTS-13 cleavage site for less than about two hours, or less than about one hour. An example of a cleavable sequence is SEQ ID NO: 1. One with skill in the art also realizes that a mutated ADAMTS-13 polypeptide, such as an ADAMTS-13 polypeptide with mutations associated with familial TTP, may be unable to cleave a functional ADAMTS-13 cleavage site.

As used herein “ADAMTS-13 gene” refers to an ADAMTS-13 nucleotide sequence. It is intended that the term interchangeably encompass fragments of the ADAMTS-13 sequence, full-length sequence, and other domains with the full-length ADAMTS-13 nucleotide sequence. Furthermore, the terms “ADAMTS-13 nucleotide sequence” or “ADAMTS-13 polynucleotide sequence” interchangeably encompass DNA, cDNA, and RNA (e.g., mRNA) sequences. One with skill in the art realizes that the ADAMTS-13 gene exists in variants in the human population, and the term ADAMTS-13 gene encompasses all such variants. Examples of such variant sequences are SEQ ID NO:9 (GenBank Accession Number NM_{—}139028), SEQ ID NO: 10 (GenBank Accession Number NM_{—}139027), SEQ ID NO:11 (GenBank Accession Number NM_{—}139026), and SEQ ID NO:12 (GenBank Accession Number NM_{—}139025).

B. Immunodetection Methods

In preferred embodiments, the present invention concerns immunodetection methods for biological components such as the A2 domain of VWF, a recombinant A2 domain of VWF, or a functional ADAMTS-13 cleavage site. As described throughout the present application, the use of wild-type and/or mutant functional ADAMTS-13 cleavage site-specific antibodies is contemplated. Immunodetection methods known to one with skill in the art include enzyme linked immunosorbent assay (ELISA), radioimmunoassay (RIA), immunoradiometric assay, fluoroimmunoassay, chemiluminescent assay, bioluminescent assay, and Western blot to mention a few. The steps of various useful immunodetection methods are well known to one with skill in the art and are described in Doolittle M H and Ben-Zeev O, 1999; Gulbis B and Galand P, 1993; De Jager R et al., 1993; and Nakamura et al., 1987, each incorporated herein by reference.

These methods include methods for purifying recombinant proteins, polypeptides and/or peptides. In these instances, the antibody removes the antigenic recombinant polypeptide from a sample. The antibody will preferably be linked to a solid support, such as in the form of a column matrix, and the sample suspected of containing recombinant protein will be applied to the immobilized antibody. The unwanted components will be washed from the column, leaving the antigen immunocomplexed to the immobilized antibody. The recombinant protein is then collected by removing it from the column.

In certain embodiments of the invention, the recombinant polypeptide that is subject to immunodetection has epitope tags incorporated into its sequence. These epitope tags may be at the N-terminus, C-terminus, or embedded within the protein sequence. These epitope tags serve as antigens for well-known and well-characterized antibodies. Such antibodies may be used in any of the immunodetection methods described herein. Examples of epitope tags contemplated in the present invention are histidine tag (MRGSHHHHHHGS SEQ ID NO:13), tag-100 (ETARFQPGYRS SEQ ID NO:14), c-myc (EQKLISEEDL SEQ ID NO:15), FLAG (DYKDDDDK SEQ ID NO:16), E2 (GVSSTSSDFRDR SEQ ID NO:17), and HA (YPYDVPDYA SEQ ID NO:18).

In terms of antigen detection, the biological sample analyzed may be any sample that is suspected of containing decreased ADAMTS-13 activity, such as a human clinical sample or specimen. Examples of appropriate samples within the scope of the present invention include blood and/or plasma from a human or other mammalian subject.

Contacting the chosen biological sample with the antibody under effective conditions and for a period of time sufficient to allow the formation of immune complexes (primary immune complexes) is generally a matter of simply adding the antibody composition to the sample and incubating the mixture for a period of time long enough for the antibodies to form immune complexes. After this time, the sample-antibody composition, such as a tissue section, ELISA plate, dot blot or western blot, will generally be washed to remove any non-specifically bound antibody species, allowing only those antibodies specifically bound within the primary immune complexes to be detected. In one embodiment, the antibody is contacted with the biological sample for two hours at 37° C. and at pH 7.4. The washes are three one minute washes with TBS, pH 7.4.

In general, the detection of immunocomplex formation is well known in the art and may be achieved through the application of numerous approaches. These methods are generally based upon the detection of a label or marker, such as radioactive, fluorescent, biological and enzymatic tags. U.S. Patents concerning the use of such labels include U.S. Pat. Nos. 3,817,837; 3,850,752; 3,939,350; 3,996,345; 4,277,437; 4,275,149 and 4,366,241, each incorporated herein by reference. Of course, one may find additional advantages through the use of a secondary binding ligand such as a second antibody and/or a biotin/avidin ligand binding arrangement, as is known in the art.

The specific antibody employed in the detection may itself be linked to a detectable label, wherein one would then simply detect this label, thereby allowing the amount of the primary immune complexes in the composition to be determined. Alternatively, the first antibody that becomes bound within the primary immune complexes may be detected by means of a second binding ligand that has binding affinity for the antibody. In these cases, the second binding ligand may be linked to a detectable label. The second binding ligand is itself often an antibody, which may thus be termed a “secondary” antibody. The primary immune complexes are contacted with the labeled, secondary binding ligand, or antibody, under effective conditions and for a period of time sufficient to allow the formation of secondary immune complexes. The secondary immune complexes are then generally washed to remove any non-specifically bound labeled secondary antibodies or ligands, and the remaining label in the secondary immune complexes is then detected. In one embodiment, the primary antibody is contacted with the secondary antibody for 45 minutes at 37° C. and at pH 7.4. The washes are three one minute washes with TBS, pH 7.4.

Further methods include the detection of primary immune complexes by a two step approach. A second binding ligand, such as an antibody, that has binding affinity for the antibody is used to form secondary immune complexes, as described above. After washing, the secondary immune complexes are contacted with a third binding ligand or antibody that has binding affinity for the second antibody, again under effective conditions and for a period of time sufficient to allow the formation of immune complexes (tertiary immune complexes). In one embodiment, the secondary antibody is contacted with the tertiary antibody for 45 minutes at 37° C. and at pH 7.4. The third ligand or antibody is linked to a detectable label, allowing detection of the tertiary immune complexes thus formed. This system may provide for signal amplification if this is desired.

In the clinical diagnosis and/or monitoring of patients with various forms of ADAMTS-13 deficiency related diseases, such as TTP, the detection of reduced cleavage of a recombinant protein comprising a functional ADAMTS13 cleavage site in comparison to the levels in a corresponding biological sample from a normal subject is indicative of a patient with TTP or other related diseases. However, as is known to those of skill in the art, such a clinical diagnosis would not necessarily be made on the basis of this method in isolation. Those of skill in the art are very familiar with differentiating between significant differences in types and/or amounts of biomarkers, which represent a positive identification, and/or low level and/or background changes of biomarkers. Indeed, background expression levels are often used to form a “cut-off” above which increased detection will be scored as significant and/or positive.

1. ELISAs

As detailed above, immunoassays, in their most simple and/or direct sense, are binding assays. Certain preferred immunoassays are the various types of enzyme linked immunosorbent assays (ELISAs) and/or radioimmunoassays (RIA) known in the art. Immunohistochemical detection using tissue sections is also particularly useful. However, it will be readily appreciated that detection is not limited to such techniques, and/or western blotting, dot blotting, FACS analyses, and/or the like may also be used.

In one exemplary ELISA, antibodies specific for one epitope tag of a recombinant protein comprising a functional ADAMTS-13 cleavage site are immobilized onto a selected surface exhibiting protein affinity, such as a well in a polystyrene microtiter plate. A test composition, such as a patient sample containing the ADAMTS-13 protein is incubated with recombinant protein comprising a functional ADAMTS-13 site. In certain embodiments of the invention, the incubating is in the range of 30 minutes to two hours. In one embodiment of the invention, the incubating is for about an hour. Then, the test composition is added to the wells. If the patient sample comprises active, wild-type ADAMTS-13, the recombinant protein is cleaved during an incubation period. After binding and/or washing to remove the cleaved portion of the recombinant polypeptide, any uncleaved recombinant protein may be detected by antibodies specific for a second epitope tag, located on the opposite side of the functional ADAMTS-13 cleavage site from the first epitope tag. The antibody to the second epitope tag is linked to a detectable label. This type of ELISA is a simple “sandwich ELISA”. Detection may also be achieved by the addition of a secondary antibody specific for the anti-epitope tag antibody, followed by the addition of a third antibody that has binding affinity for the second antibody, with the third antibody being linked to a detectable label.

Irrespective of the format employed, ELISAs have certain features in common, such as coating, incubating and binding, washing to remove non-specifically bound species, and detecting the bound immune complexes. These are described below.

In coating a plate with either antigen or antibody, one will generally incubate the wells of the plate with a solution of the antigen or antibody, either overnight or for a specified period of hours. The wells of the plate will then be washed to remove incompletely adsorbed material. Any remaining available surfaces of the wells are then “coated” with a nonspecific protein that is antigenically neutral with regard to the test antisera. These include bovine serum albumin (BSA), casein or solutions of milk powder. The coating allows for blocking of nonspecific adsorption sites on the immobilizing surface and thus reduces the background caused by nonspecific binding of antisera onto the surface.

In ELISAs, it is probably more customary to use a secondary or tertiary detection means rather than a direct procedure. Thus, after binding of a protein or antibody to the well, coating with a non-reactive material to reduce background, and washing to remove unbound material, the immobilizing surface is contacted with the biological sample to be tested under conditions effective to allow immune complex (antigen/antibody) formation. Detection of the immune complex then requires a labeled secondary binding ligand or antibody, and a secondary binding ligand or antibody in conjunction with a labeled tertiary antibody or a third binding ligand. In one embodiment, the antigen, the recombinant protein, is contacted with the primary antibody for 45 minutes at 37° C. and at pH 7.4. The primary antibody is contacted with the secondary antibody for 45 minutes at 37° C. and at pH 7.4. The washes are three one minute washes with TBS, pH 7.4.

“Under conditions effective to allow immune complex (antigen/antibody) formation” means that the conditions preferably include diluting the antigens and/or antibodies with solutions such as BSA, bovine gamma globulin (BGG) or phosphate buffered saline (PBS)/Tween. These added agents also tend to assist in the reduction of nonspecific background.

The “suitable” conditions also mean that the incubation is at a temperature or for a period of time sufficient to allow effective binding. Incubation steps are typically from about 1 to 2 to 4 hours or so, at temperatures preferably on the order of 25° C. to 27° C., or may be overnight at about 4° C.

Following all incubation steps in an ELISA, the contacted surface is washed so as to remove non-complexed material. A preferred washing procedure includes washing with a solution such as PBS/Tween, or borate buffer. Following the formation of specific immune complexes between the test sample and the originally bound material, and subsequent washing, the occurrence of even minute amounts of immune complexes may be determined.

To provide a detecting means, the second or third antibody will have an associated label to allow detection. Preferably, this will be an enzyme that will generate color development upon incubating with an appropriate chromogenic substrate. Thus, for example, one will desire to contact or incubate the first and second immune complex with a urease, glucose oxidase, alkaline phosphatase or hydrogen peroxidase-conjugated antibody for a period of time and under conditions that favor the development of further immune complex formation (e.g., incubation for 2 hours at room temperature in a PBS-containing solution such as PBS-Tween).

After incubation with the labeled antibody, and subsequent to washing to remove unbound material, the amount of label is quantified, e.g., by incubation with a chromogenic substrate such as urea, or bromocresol purple, or 2,2′-azino-di-(3-ethyl-benzthiazoline-6-sulfonic acid (ABTS), or H₂O₂, in the case of peroxidase as the enzyme label. Quantification is then achieved by measuring the degree of color generated, e.g., using a visible spectra spectrophotometer.

2. Gel Chromatography and Immunoblotting

In certain embodiments of the invention, the recombinant polypeptide comprising a functional ADAMTS-13 site may be detected through gel chromatography or immunoblotting. Common gels are dextran, agarose and polyacrylamide.

In certain embodiments of the invention, a patient sample is contacted with a recombinant polypeptide comprising a functional ADAMTS-13 site. The proteins in the sample are separated by gel electrophoresis. ADAMTS-13 activity is detected by detecting the appearance of a cleavage band of the recombinant polypeptide. In certain embodiments of the invention, the detecting is accomplished through immunoblotting. In a specific embodiment of the invention, the detecting is through staining of an SDS-PAGE gel, for example with a Coomassie blue stain.

C. Antibody Conjugates

The present invention further provides antibodies useful for the detection of ADAMTS-13 activity, generally of the monoclonal type, that are linked to at least one agent to form an antibody conjugate. In one embodiment of the invention, said antibodies are directed towards the A2 domain of VWF. In specific embodiments of the invention, the antibodies are directed towards recombinant A2 domain of VWF. In another embodiment of the invention, the antibodies are directed to markers operatively linked to the recombinant A2 domain of VWF. In order to increase the efficacy of antibody molecules as diagnostic or therapeutic agents, it is conventional to link or covalently bind or complex at least one desired molecule or moiety. Such a molecule or moiety may be, but is not limited to, at least one effector or reporter molecule. Effector molecules comprise molecules having a desired activity, e.g., cytotoxic activity. Non-limiting examples of effector molecules which have been attached to antibodies include toxins, anti-tumor agents, therapeutic enzymes, radio-labeled nucleotides, antiviral agents, chelating agents, cytokines, growth factors, and oligo- or poly-nucleotides. By contrast, a reporter molecule is defined as any moiety which may be detected using an assay. Non-limiting examples of reporter molecules which have been conjugated to antibodies include enzymes, radiolabels, haptens, fluorescent labels, phosphorescent molecules, chemiluminescent molecules, chromophores, luminescent molecules, photoaffinity molecules, colored particles or ligands, such as biotin.

Any antibody of sufficient selectivity, specificity or affinity may be employed as the basis for an antibody conjugate. Such properties may be evaluated using conventional immunological screening methodology known to those of skill in the art. Sites for binding to biological active molecules in the antibody molecule, in addition to the canonical antigen binding sites, include sites that reside in the variable domain that can bind pathogens, B-cell superantigens, the T cell co-receptor CD4 and the HIV-1 envelope (Sasso et al., 1989; Shorki et al., 1991; Silvermann et al., 1995; Cleary et al., 1994; Lenert et al., 1990; Berberian et al., 1993; Kreier et al., 1991). In addition, the variable domain is involved in antibody self-binding (Kang et al., 1988), and contains epitopes (idiotopes) recognized by anti-antibodies (Kohler et al., 1989).

Certain examples of antibody conjugates are those conjugates in which the antibody is linked to a detectable label. “Detectable labels” are compounds and/or elements that can be detected due to their specific functional properties, and/or chemical characteristics, the use of which allows the antibody to which they are attached to be detected, and/or further quantified if desired. Another such example is the formation of a conjugate comprising an antibody linked to a cytotoxic or anti-cellular agent, and may be termed “immunotoxins”.

Antibody conjugates are generally preferred for use as diagnostic agents. Antibody diagnostics generally fall within two classes, those for use in in vitro diagnostics, such as in a variety of immunoassays, and/or those for use in vivo diagnostic protocols, generally known as “antibody-directed imaging”. Many appropriate imaging agents are known in the art, as are methods for their attachment to antibodies (see, for e.g., U.S. Pat. Nos. 5,021,236; 4,938,948; and 4,472,509, each incorporated herein by reference). The imaging moieties used can be paramagnetic ions; radioactive isotopes; fluorochromes; NMR-detectable substances; X-ray imaging.

In the case of paramagnetic ions, one might mention by way of example ions such as chromium (III), manganese (II), iron (III), iron (II), cobalt (II), nickel (II), copper (II), neodymium (III), samarium (III), ytterbium (III), gadolinium (III), vanadium (II), terbium (III), dysprosium (III), holmium (III) and/or erbium (III), with gadolinium being particularly preferred. Ions useful in other contexts, such as X-ray imaging, include but are not limited to lanthanum (III), gold (III), lead (II), and especially bismuth (III).

In the case of radioactive isotopes for therapeutic and/or diagnostic application, one might mention astatine²¹¹, ¹⁴carbon, ⁵¹chromium, ³⁶chlorine, ⁵⁷cobalt, ⁵⁸cobalt, copper⁶⁷, ¹⁵²Eu, gallium⁶⁷, ³hydrogen, iodine¹²³, iodine¹²⁵, iodine¹³¹, indium¹¹¹, ⁵⁹iron, ³²phosphorus, rhenium¹⁸⁶, rhenium¹⁸⁸, ⁷⁵selenium, ³⁵sulphur, technicium^{99m} and/or yttrium⁹⁰. ¹²⁵I is often being preferred for use in certain embodiments, and technicium^{99m} and/or indium¹¹¹ are also often preferred due to their low energy and suitability for long range detection. Radioactively labeled monoclonal antibodies of the present invention may be produced according to well-known methods in the art. For instance, monoclonal antibodies can be iodinated by contact with sodium and/or potassium iodide and a chemical oxidizing agent such as sodium hypochlorite, or an enzymatic oxidizing agent, such as lactoperoxidase. Monoclonal antibodies according to the invention may be labeled with technetium⁹⁹m by ligand exchange process, for example, by reducing pertechnate with stannous solution, chelating the reduced technetium onto a Sephadex column and applying the antibody to this column. Alternatively, direct labeling techniques may be used, e.g., by incubating pertechnate, a reducing agent such as SNCl₂, a buffer solution such as sodium-potassium phthalate solution, and the antibody. Intermediary functional groups which are often used to bind radioisotopes which exist as metallic ions to antibody are diethylenetriaminepentaacetic acid (DTPA) or ethylene diaminetetracetic acid (EDTA).

Among the fluorescent labels contemplated for use as conjugates include Alexa 350, Alexa 430, AMCA, BODIPY 630/650, BODIPY 650/665, BODIPY-FL, BODIPY-R6G, BODIPY-TMR, BODIPY-TRX, Cascade Blue, Cy3, Cy5,6-FAM, Fluorescein Isothiocyanate (FITC), HEX, 6-JOE, Oregon Green 488, Oregon Green 500, Oregon Green 514, Pacific Blue, REG, Rhodamine Green, Rhodamine Red, Renographin, ROX, TAMRA, TET, Tetramethylrhodamine, and/or Texas Red.

Another type of antibody conjugates contemplated in the present invention are those intended primarily for use in vitro, where the antibody is linked to a secondary binding ligand and/or to an enzyme (an enzyme tag) that will generate a colored product upon contact with a chromogenic substrate. Examples of suitable enzymes include urease, alkaline phosphatase, (horseradish) hydrogen peroxidase or glucose oxidase. Preferred secondary binding ligands are biotin and/or avidin and streptavidin compounds. The use of such labels is well known to those of skill in the art and are described, for example, in U.S. Pat. Nos. 3,817,837; 3,850,752; 3,939,350; 3,996,345; 4,277,437; 4,275,149 and 4,366,241; each incorporated herein by reference.

Molecules containing azido groups may also be used to form covalent bonds to proteins through reactive nitrene intermediates that are generated by low intensity ultraviolet light (Potter & Haley, 1983). In particular, 2- and 8-azido analogues of purine nucleotides have been used as site-directed photoprobes to identify nucleotide binding proteins in crude cell extracts (Owens & Haley, 1987; Atherton et al., 1985). The 2- and 8-azido nucleotides have also been used to map nucleotide binding domains of purified proteins (Khatoon et al., 1989; King et al., 1989; and Dholakia et al., 1989) and may be used as antibody binding agents.

Several methods are known in the art for the attachment or conjugation of an antibody to its conjugate moiety. Some attachment methods involve the use of a metal chelate complex employing, for example, an organic chelating agent such a diethylenetriaminepentaacetic acid anhydride (DTPA); ethylenetriaminetetraacetic acid; N-chloro-p-toluenesulfonamide; and/or tetrachloro-3α-6α-diphenylglycouril-3 attached to the antibody (U.S. Pat. Nos. 4,472,509 and 4,938,948, each incorporated herein by reference). Monoclonal antibodies may also be reacted with an enzyme in the presence of a coupling agent such as glutaraldehyde or periodate. Conjugates with fluorescein markers are prepared in the presence of these coupling agents or by reaction with an isothiocyanate. In U.S. Pat. No. 4,938,948, imaging of breast tumors is achieved using monoclonal antibodies and the detectable imaging moieties are bound to the antibody using linkers such as methyl-p-hydroxybenzimidate or N-succinimidyl-3-(4-hydroxyphenyl)propionate.

In other embodiments, derivatization of immunoglobulins by selectively introducing sulfhydryl groups in the Fc region of an immunoglobulin, using reaction conditions that do not alter the antibody combining site are contemplated. Antibody conjugates produced according to this methodology are disclosed to exhibit improved longevity, specificity and sensitivity (U.S. Pat. No. 5,196,066, incorporated herein by reference). Site-specific attachment of effector or reporter molecules, wherein the reporter or effector molecule is conjugated to a carbohydrate residue in the Fc region have also been disclosed in the literature (O'Shannessy et al., 1987). This approach has been reported to produce diagnostically and therapeutically promising antibodies which are currently in clinical evaluation.

D. Immunodetection Kits

In still further embodiments, the present invention concerns immunodetection kits for use with the immunodetection methods described above. Antibodies specific for a recombinant polypeptide comprising a functional ADAMTS-13 cleavage site will preferably be included in the kit. The immunodetection kits will thus comprise, in suitable container means, a first antibody that binds to any region on the recombinant polypeptide on the N-terminal side of the functional ADAMTS-13 cleavage site. The kit will also comprise a second antibody that binds to any region on the recombinant polypeptide on the C-terminal side of the functional ADAMTS-13 cleavage site. The kit may comprise an antibody specific for a first epitope tag and a second epitope tag of the recombinant polypeptide.

In preferred embodiments, monoclonal antibodies will be used. In certain embodiments, one antibody used may be pre-bound to a solid support, such as a column matrix and/or well of a microtiter plate.

The immunodetection reagents of the kit may take any one of a variety of forms, including those detectable labels that are associated with and/or linked to the given antibody. Detectable labels that are associated with and/or attached to a secondary binding ligand are also contemplated. Exemplary secondary ligands are those secondary antibodies that have binding affinity for the first antibody.

Further suitable immunodetection reagents for use in the present kits include the two-component reagent that comprises a secondary antibody that has binding affinity for the first antibody, along with a third antibody that has binding affinity for the second antibody, the third antibody being linked to a detectable label. As noted above, a number of exemplary labels are known in the art and/or all such labels may be employed in connection with the present invention.

The kits may further comprise a suitably aliquoted composition of recombinant polypeptide comprising a functional ADAMTS-13 cleavage site, whether labeled and/or unlabeled, as may be used to prepare a standard curve for a detection assay. The kits may contain antibody-label conjugates either in fully conjugated form, in the form of intermediates, and/or as separate moieties to be conjugated by the user of the kit. The components of the kits may be packaged either in aqueous media and/or in lyophilized form.

The container means of the kits will generally include at least one vial, test tube, flask, bottle, syringe and/or other container means, into which the antibody may be placed, and/or preferably, suitably aliquoted. The kits of the present invention will also typically include a means for containing the antibody, antigen, and/or any other reagent containers in close confinement for commercial sale. Such containers may include injection and/or blow-molded plastic containers into which the desired vials are retained.

E. Decreased ADAMTS-13 Activity and Disease

In certain embodiments of the invention, decreased activity of ADAMTS-13 cleavage of a recombinant polypeptide correlates to increased likelihood of the development of certain diseases, such as TTP. The term “correlating,” as used herein in reference to the use of prognostic indicators to determine a prognosis, refers to comparing the presence or amount of the prognostic indicator in a patient to its presence or amount in (i) persons known to suffer from, or known to be at risk of, a given condition; or (ii) in persons known to be free of a given condition; or (iii) both. For example, a decreased level of ADAMTS-13 activity in a patient can be compared to a level known to be associated with an increased predisposition for development of TTP. The patient's ADAMTS-13 activity level is said to have been correlated with a prognosis; that is, the skilled artisan can use the patient's ADAMTS-13 activity level to determine the likelihood that the patient is at risk for TTP, and respond accordingly. Alternatively, the patient's ADAMTS-13 activity level can be compared to a ADAMTS-13 activity level known to be associated with a good outcome (e.g., no risk of developing TTP), and determine if the patient's prognosis is predisposed to the good outcome. A “baseline ADAMTS-13 activity” level is the ADAMTS-13 activity level before a specific event, such as aging.

“Predictive value” is the probability that a mammal with a positive test has the disease and one with a negative test result does not have the disease. It may be determined by the prevalence of the condition and the sensitivity and specificity of the test. It can be expressed in two ways as either the positive predictive value and the negative predictive value. Prevalence is a measure of the incidence of disease in a population at one given time, e.g. cases per 100,000. In certain embodiments, one or more additional prognostic indicators can be combined with a level of ADAMTS-13 activity in a patient sample to increase the predictive value of ADAMTS-13 activity as a prognostic indicator. The phrase “increases the predictive value” refers to the ability of two or more combined prognostic indicators to improve the ability to predict a given outcome, in comparison to a prediction obtained from any of the prognostic indicators alone.

The sensitivity of a diagnostic test is a measure of the proportion of mammals with a disease, for example TTP, having a positive test result. The specificity of a diagnostic test is a measure of proportion of mammals without a disease having a negative test result.

Operating characteristics of diagnostic tests and procedures are measures of the technical performance of these technologies. A means of expressing these values of the diagnostic test of the present invention is with a receiver operating characteristic (ROC) curve, which plots the relationship between the true positive ratio, the sensitivity, and false positive ratio (1—specificity) as a function of the cutoff level of a disease marker. ROC curves help to demonstrate how raising or lowering the cutoff point for defining a positive test result affects tradeoffs between correctly identifying people with a disease (true positives) and incorrectly labeling a person as positive who does not have the condition (false positives).

In accordance with standard practice, ADAMTS-13 activity levels can be measured against minimum clinically effective threshold values. The “diagnostic accuracy” or “clinical efficacy” of a test, assay, or method concerns the ability of the test, assay, or method to distinguish between patients having a disease, condition, or syndrome and patients not having that disease, condition, or syndrome based on whether the patients have a “clinically significant presence” of decreased ADAMTS-13 activity. By “clinically significant presence” is meant that the ADAMTS-13 activity level in the patient sample is less than the predetermined cutoff point, or “threshold value.” The term “clinically effective threshold value” refers to an ADAMTS-13 activity level less than a predetermined threshold value. Changing the cutoff point, or threshold value of a test, changes the sensitivity and specificity in a qualitatively inverse relationship. For example, if the threshold value is raised, more individuals in the population tested will typically have test results under the cut point or threshold value. However, at the same time, there will be more false positives because more people who do not have the disease, condition, or syndrome will be indicated by the test to have relative ADAMTS-13 activity level below the threshold value and therefore to be reported as positive rather than being correctly indicated by the test to be negative. Accordingly, the specificity of the test will be decreased. Similarly, lowering the cutoff point will tend to decrease the sensitivity and increase the specificity. Therefore, in assessing the accuracy and usefulness of a proposed medical test, assay, or method for assessing a patient's condition, one should always take both sensitivity and specificity into account and be mindful of what the threshold value is at which the sensitivity and specificity are being reported because sensitivity and specificity may vary significantly over the range of threshold values. One with skill in the art realizes that in certain situations, such as screening, it is desirable to increase the specificity and decrease the sensitivity of the test by lowering the threshold value of ADAMTS-13 activity level. In other embodiments, it may be desirable to increase the sensitivity and decrease the specificity of the test by increasing the threshold value. In certain embodiments of the invention, the sensitivity of the invention(ADAMTS-13 activity assay) in detecting TTP, or other diseases related to ADAMTS-13 deficiency, is within the range of at least 80%.

### EXAMPLES

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1

### Antibodies and Isolated ADAMTS-1

Ascites containing mouse monoclonal antibody VP-1 directed against amino acids residues 1591-1605 of SEQ ID NO:8, VWF protein,was a gift of Dr. Z. M. Ruggeri (Scripps Research Institute, La Jolla, Calif.). Antibody VP-1 was further purified by using a protein G column. Similarly, autoantibody against ADAMTS-13 purified from plasma of an acquired TTP patient by using a protein G column. A total protein concentration of 60 μg/mL was obtained. The partially isolated ADAMTS-13 was obtained as previously described (see Dong et al., 2002) and the total protein concentration was 275. μg/mL.

### Example 2

### Construction of a Recombinant Single-Tagged VWF-A2 Peptide

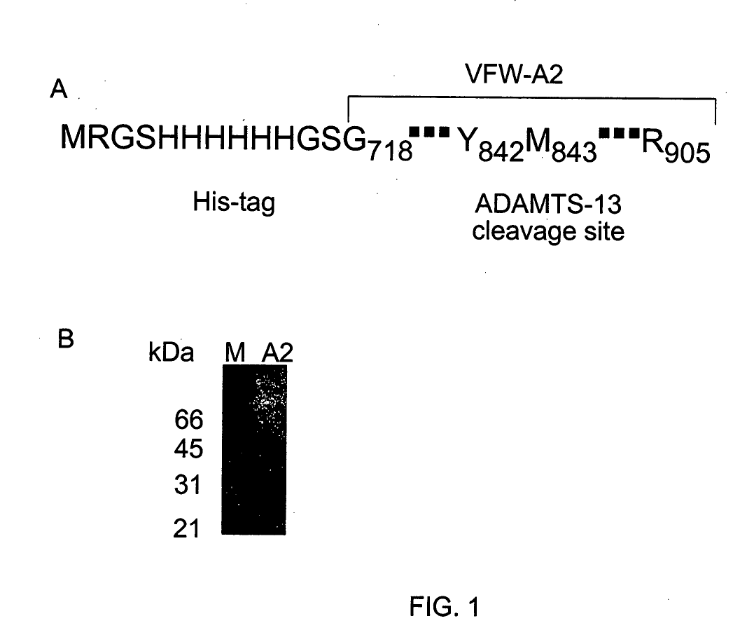

Complementary DNA encoding the human VWF-A2 domain (aa 1481-1668) was generated by polymerase chain reaction (PCR) using full-length human VWF cDNA as a template. The PCR end primers were designed to introduce a BamHI restriction site at the 5′ end (AAGGCCGGATCCGGGCTCTTGGGGGTTTCG SEQ ID NO: 19) and HindIII restriction site at the 3′ end (AAGGCCAAGCTTCCTCTGCAGCACCAGGTC SEQ ID NO: 20). The PCR product was digested with BamHI and HindIII restriction enzymes and inserted into pQE9 (Qiagen, Chatsworht, Calif.) for expression in E. coli. The recombinant VWF-A2 domain was expressed as a His-tag fusion protein containing 12 residues at the N terminus from the expression vector (MRGSHHHHHHGS SEQ ID NO: 13) (FIG. 1). The recombinant single-tagged construct insert has a nucleotide sequence corresponding to SEQ ID NO: 21

### Example 3

### Construction of a Recombinant Double-Tagged VWF-A2 Peptide

Complementary DNA encoding the human VWF-A2 domain (SEQ ID NO:2) was generated by polymerase chain reaction (PCR) using full-length human VWF cDNA as a template. The PCR end primers were designed to introduce a BamHI restriction site at the 5′ end (AAGGCCGGATCCGGGCTCTTGGGGGTTTCG SEQ ID NO:22) and Sall restriction site at the 3′ end (AAGGCCGTCGACTCCTCTGCAGCACCAGGTC SEQ ID NO:23). The PCR product was digested with BamHI and Sall restriction enzymes and inserted into pQE-100 double tag vector (Qiagen, Chatsworth, Calif., USA) for expression in Escherichia coli. The recombinant VWF-A2 domain was expressed with the 6×His tag at the N-terminus (MRGSHHHHHHGS SEQ ID NO:13) and the Tag-100 epitope (ETARFQPGYRS SEQ ID NO:14) at the C-terminus from the expression vector. The recombinant double-tagged construct has a nucleotide sequence insert corresponding to SEQ ID NO: 21

### Example 4

### Expression and Purification of the Recombinant Single and Double-Tagged VWF-A2 Peptide

Escherichia coli M15 (pREP4) (Qiagen) containing the pQE9-VWF-A2 domain or the pQE100-VWF-A2 domain were cultured, induced and lysed as described previously. For purification, the washed pellet was solubilized in 7.5 M urea in 50 mM Tris-HCl pH 7.5. The solubilized protein was passed over a Co2+-chelated Sepharose (TALON Superflow; Clontech, Palo Alto, Calif., USA) column equilibrated with 5M urea, 50 mM Tris-HCl, 500 mM NaCl, pH7.4 buffer. The VWF-A2 peptide was eluted from the column with 150 mM imidazole. The buffer was rapidly exchanged with 25 mM Tris-HCl, 150 mM NaCl, 0.05% Tween-20, pH 7.4 (TBS-T) using a desalting column (Amersham). Protein concentration was determined by the BCA method (Pierce Chemical Co., Rockford, Ill., USA).

### Example 5

### Measurement of ADAMTS-13 Activity in Plasma by ELISA

Plasma samples from healthy donors or patients with TTP were collected for the studies. Blood was drawn under a protocol approved by the Institutional Review Board for Human Subject Research at the Baylor College of Medicine and Affiliated Hospitals. Samples were also obtained from patients who had been clinical diagnosed as familial adult acquired idiopathic TTP. The diagnosis of TTP was based on profound thrombocytopenia (platelet<:30000 μL⁻¹, schistocytosis, extremely high levels of lactate dehydrogenase (LDH), variable neurological findings, and subcutaneous and mucus bleedings. Furthermore, all patients responded to either plasmapheresis (familial TTP) or plasma exchange (adult acquired idiopathic TTP).

Samples of either citrated (0.38% sodium citrate) or PPACK (75 μM) normal plasma or plasma from patients with TTP (either acquired TTP with very low ADAMTS-13 activity as a result of an inhibitor, or congenital TTP with near-absent ADAMTS-13 activity) were diluted in 25 mM Tris-HCl, 150 mM NaCl pH 7.4 (TBS) and mixed with recombinant single-tagged VWF-A2 peptide (30μg/mL in TBS) or recombinant double-tagged VWF-A2 peptide (5 μg/mL in TBS) to measure ADAMTS-13 activity in plasma. For the recombinant double-tagged VWF-A2 peptide, three different dilutions of plasma (1:1, 1:5, and 1:50) from both healthy controls (n=39) and TTP patients (n=16) were tested. The mixture was added into microtiter wells (80 μL per well) coated with Ni²⁺ (Ni-NTA HisSorb Strips; Qiagen) and incubated for 2 h at 37° C. In addition, congenital TTP plasma was mixed 1:1 with normal plasma and incubated with the recombinant A2 peptides (single or double-tagged) to demonstrate that the ADAMTS-13 from the normal plasma restores cleavage activity. The assay was performed at conditions of 1:1 dilution plasma.

The VWF-A2 single-tagged recombinant peptide (30 μg/mL) was incubated with VP-1 (80 μg/mL) at 37° C. for 15 minutes to study the effect of the monoclonal antibody, VP-1 on VWF-A2 cleavage by ADAMTS-13. Then the sample was mixed with plasma or isolated enzyme.

A range of dilutions of partially isolated ADAMTS-13 enzyme from plasma (275 μg/mL total protein) was also incubated with the recombinant double-tagged VWF-A2 peptide to assure that isolated ADAMTS-13 specifically was responsible for the observed cleavage of recombinant VWF-A2 peptide. After incubation for 2 h, the wells were washed three times (approximately 1 min per wash) with TBS, and monoclonal antibody Tag-100 (against Tag-100) (Qiagen) (1:2000 in TBS) was added and incubated for one additional hour at 37° C. The wells were then washed four times with TBS and incubated with a 1:3000 dilution of goat peroxidase-conjugated monoclonal antimouse IgG antibody (Sigma, St Louis, Mo., USA) for 45 min at 37° C. The wells were washed four times, and the substrate (o-phenylenediamine; Sigma) was added. After 10-15 min of substrate conversion, reactions were stopped with 0.025 mL of 2 NH₂SO₄, and the plates were read at 490 nm.

In each Ni²⁺-coated microtiter plate, TBS containing A2 protein was incubated in wells to obtain the value of captured VWF-A2 substrate. This absorbance value represents the undigested VWF-A2. For background, wells were incubated with the diluted plasma without VWF-A2 peptide. Thus, to summarize binding data in the percentage of ADAMTS-13 activity, optical density (OD) (490 nm) values obtained for each sample at specific plasma dilution were converted as follows: [1-(OD sample-OD Bkgd/OD A2 only-OD Bkgd)]×100=activity percentage

### Example 6

### Electrophoresis and Western Blotting

The ability of ADAMTS-13 to cleave the purified recombinant single-tagged VWF-A2 domain was determined. Enzyme activity was detected by visualizing a band corresponding to a molecular weight of 16 kDa (SEQ ID NO: 2 operably linked to a His-tag). After one hour incubation, proteolysis by plasma from a healthy individual and the partially isolated enzyme produced the expected 16 kD band corresponding to the sequence 1481-1605 of SEQ ID NO:8 plus the 12 amino acid residues from the His-tag. See FIG. 1A. Identical results were obtained with normal plasma anticoagulated with either PPACK or sodium citrate. The protein was analyzed under non-reducing conditions.

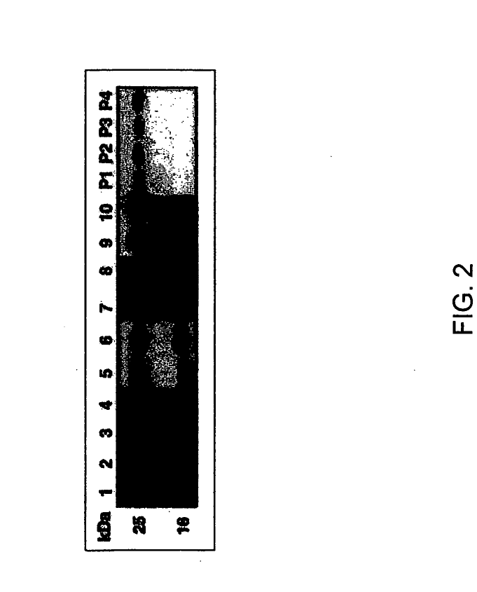

The cleavage of VWF-A2 protein was efficiently detected by immunoblotting, as demonstrated in FIG. 2. Healthy plasma (FIG. 2, lane 1), and partially isolated ADAMTS-13 (FIG. 2, lane 3) produced the expected band of 16 kDa. The enzyme activity was inhibited with the addition of the monoclonal antibody VP-1 (80 μg/mL ) to the mixture of healthy plasma FIG. 2, (lane 2) or isolated ADAMTS-13 (FIG. 2, lane 4) with A2 protein. The poor cleavage activity present in plasma from a congenital TTP patient (FIG. 2, lane 5) was restored when isolated ADAMTS-13 was mixed (1:1) with the patient plasma (FIG. 2, lane 6). In addition, both healthy plasma (FIG. 2, lane 7) and partially isolated ADAMTS-13 (FIG. 2, lane 8) failed to generate the 16 kDa fragment in the presence of EDTA (5 mM). The activity was effectively blocked when healthy plasma was mixed (1:1) with either isolated autoantibody against ADAMTS-13 (FIG. 2, lane 9) or plasma from an acquired TTP patient (FIG. 2, lane 10) and incubated with A2 protein. P1 and P2 show plasma from acquired TTP patients incubated with A2. P3 and P4 show plasma from congenital TTP patients incubated with A2. The monoclonal antibody VP-1 effectively blocked proteolysis (FIG. 2, lanes 2 and 4) when it was added to normal plasma or isolated enzyme. Furthermore, the poor cleavage activity present in a congenital plasma sample (FIG. 2, lane 5) was increased. The samples (2 μL) were mixed with non-reduced sample buffer (10 μL) and the proteins separated by 12% SDS-PAGE (45 min. at 180 volts). The proteins were transferred into a polyvinylidine difluoride (PVDF, Waters, Milford, Mass.) membrane at 15 volts for 15 min. The membrane was incubated with 5% non-fat dry milk in TBS-T for 30 min. After washing the membrane one time with TBS-T, monoclonal anti-histidine tag (Sigma) diluted in TBS-T (1:5,000) was added to the membrane and incubated for 30 min. at RT. The bands were visualized with enhanced chemiluminescence (Pierce).

### Example 7

### Endothelial Cell Culture and Preparation of UL VWF

Endothelial cells were obtained from human umbilical veins (HUVEC) as described previously and used for ULVWF string assay and as the source of ULVWF. The umbilical cords were first washed with phosphate buffer (140 mM NaCl, OAmMKCl, 1.3 mM NaH2PO4, 1.0 mM Na₂HPO₄, 0.2% glucose pH 7.4) and then infused with a collagenase solution (0.02%; Invitrogen Life Technologies, Carlsbad, Calif., USA). After a 30-min incubation at room temperature, the cords were rinsed with 100 mL of the phosphate buffer. Eluates containing endothelial cells were centrifuged at 250×g for 10 min. The cell pellets were resuspended in Medium 199 (Invitrogen Life Technologies) containing 20% heat-inactivated fetal calf serum and 0.2 mM of L-glutamine. The endothelial cells were then plated on a culture dish coated with 1% gelatin and grown until confluent (3-5 days).

To obtain ULVWF, confluent HUVEC cultures were first washed with PBS and incubated with a serum-free medium (insulin 5-10 μg/mL transferrin 5 μg/mL, M199, 1% glutamine) for 48-72 h. The cells were then treated with 100 μM histamine for 30 min at 37° C. to induce the release of ULVWF. After incubation, the conditioned medium was collected and centrifuged at 150×g for 10 min to remove cell debris and the supernatant was used as the source of ULVWF multimers. The multimeric composition of purified plasma VWF and ULVWF was evaluated by SDS-1% agarose gel electrophord,is and chemiluminescence.

### Example 8

### Measurement of ADAMTS-13 Activity in Immunoblotting Techniques

Two immunoblotting methods were used to verify the results from the ELISA assay. Assay 1 uses ULVWF multimers (VWF:Ag=15±2%) from the supernatant of cultured HUVEC in place of smaller, plasma-type VWF forms. ADAMTS-13 activity is estimated by the disappearance of UL and large VWF multimers in the presence of test plasma, compared with the presence of dilutions of pooled normal plasma. ADAMTS-13 activity in either normal plasma (NP) or TTP plasma (each diluted 1:5 in 10 mM Tris-Cl, 150 mM NaCl buffer pH 8.0) is determined by mixing each plasma sample with HUVEC supernatant (containing ULVWF multimers), 1.5 M urea, and 10 mM BaC12 at pH 8 for 24 h at 37° C. As one negative control (no ADAMTS-13 activity), buffer is substituted for diluted normal plasma in the mixture; as a second negative control, EDTA is added to the normal plasma mixture before incubation. Electrophoresis of non-reduced SDS samples is into 1% agarose, and VWF multimers are displayed by rabbit polyclonal antihuman VWF antibody and chemiluminescence. Pooled normal plasma sample has, by definition, 100% ULVWF multimer cleaving capacity (ADAMTS-13 activity). Most of the TTP patient plasma samples studied have <5% ADAMTS-13 activity.

Assay 2 was done under static conditions using the single tag recombinant VWF-A2 as substrate. Ten-microliter samples of PPACK (75 μM) plasma were incubated with the single tag VWF-A2 peptide (30 μg/mL⁻¹ in TBS) at 37° C. (total volume 25 μL). Two microliters from the mixture were collected after 1 h incubation and added to 10 μL of non-reduced sample buffer. Cleavage was assessed by electrophoresis and immunoblotting. Densitometer quantification (Scientific Imaging Systems; Kodak) of the 16-kDa band [ratio of 16-kDa band to total A2 bands (16 kDa+25 kDa) in arbitrary units] was performed in order to obtain relative cleavage activity of the ADAMTS-13 on the A2 protein.

### Example 9

### Measurement of ADAM TS-13 Activity in Plasma Under Flow

The formation and cleavage of ULVWF strings by ADAMTS-13 in test plasma were studied under flow in a parallel-plate flow chamber system and observed by phase-contrast video microscopy. The endothelial cells were grown as a monolayer on the coverslip that formed the bottom of a parallel-plate flow chamber. The chamber was kept at 37° C. with a thermostatic air bath during the experiments. The assembled parallel-plate flow chamber was mounted onto an inverted-stage microscope (Eclipse TE300; Nikon, Garden City, N.Y., USA) equipped with a high-speed digital camera (Model Quantix; Photometrics, Tucson, Ark., USA). A syringe pump connected to the outlet port draws a washed platelet suspension through the chamber at a defined flow rate to generate a wall shear stress of 2.5 dyncm-2. The formation and cleavage of ULVWF strings were recorded using a digital camera and acquired images were analyzed offline using MetaMorph software (Universal Images, West Chester, Pa., USA). The formation and cleavage of ULVWF multimeric strings were quantitated in 20 continuous 40× view-fields over the course of 2-5 min.

The soluble double-tagged VWF-A2 peptide was identically purified by the procedures previously used to isolate the singletag A2 peptide. The yield of purified double-tagged VWF-A2 peptide was 1-2 μg/L⁻¹ of bacterial culture, with a calculated molecular mass of 23.7 kDa. The protein does not contain cysteine residues, in good agreement with the 27 kDa molecular mass estimated by non-reducing SDS-PAGE.

The cleavage of the double-tagged VWF-A2 peptide by ADAMTS-13 was efficiently detected by immunoblotting, as previously demonstrated using the single-tag VWF-A2 peptide. The addition of the Tag-100 epitope in the C-terminal of the VWF-A2 peptide does not change its cleavage susceptibility.

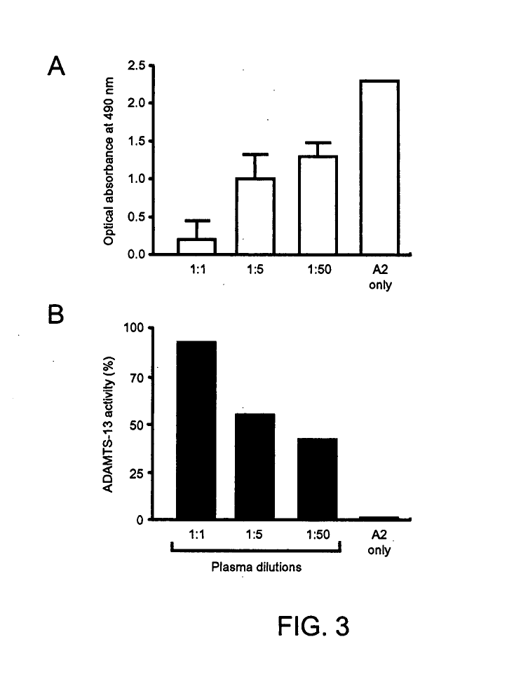

In each Ni²⁺-coated microtiter plate, TBS containing A2 protein was incubated in at least three wells to obtain the mean value of captured VWF-A2 substrate. This value was defined as 100% undigested VWF-A2. For background, wells were incubated with the diluted plasma without VWF-A2 peptide. Thus, cleavage of VWF-A2 peptide was defined by the reduction of absorbance (FIG. 3A). The absorbance was then converted into the percentage of ADAMTS-13 activity using equation 1 described above (FIG. 3B).

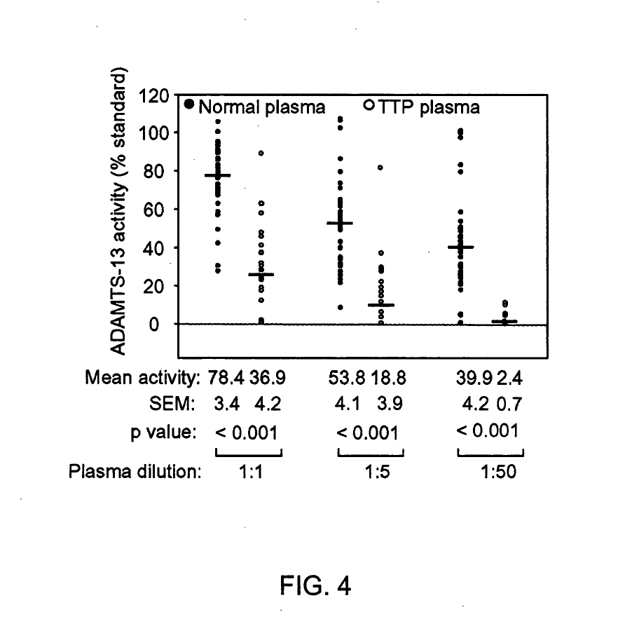

Using this new ELISA method, the difference in mean value between normal (n=39) and TTP subjects (n=16) was significant (FIG. 4). Three different plasma dilutions were used to measure ADAMTS-13 activity in plasma from normal and TTP patients. As the panel shows, a highly significant difference was obtained in the mean of enzyme activity between the normals and patients in all the dilutions tested. The highest plasma dilution tested (1:50), proved to be the most sensitive as 13 (81%) of the 16 TTP samples had <6% activity. In contrast, with the exception of three (7.7%) that had <5% activity, 36 normals had an enzyme activity of >17%. For each sample, the value represents the average of triplicate experiments. The measurements were made at three different plasma dilutions with TBS buffer. Four patients who had clinically been diagnosed as TTP showed ADAMTS-13 activity >50% at 1:1 dilution, but ADAMTS-13 deficiency became apparent at 1:50 dilution of plasma (FIG. 4). Furthermore, at this plasma dilution three normal individuals had enzyme activity <5%.

Pooled nornal plasma (n=25 donors) was analyzed 10 times and the coefficient of variation (CV) measured to be approximately 2.4%.

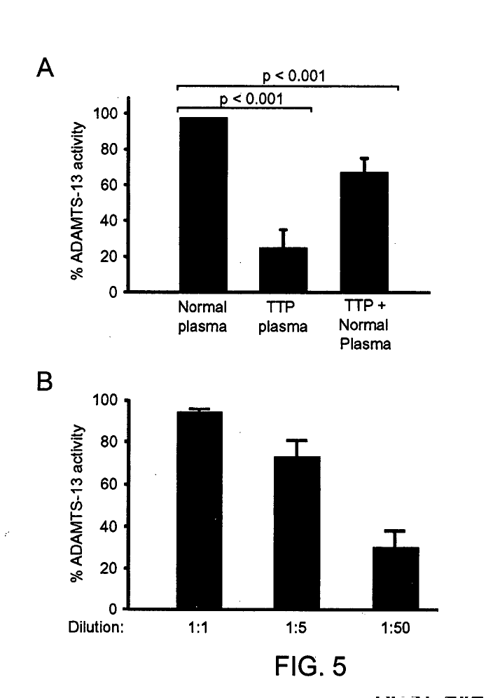

The specificity ofADAMTS-13 cleavage of the recombinant VWF-A2 was further determined by two means. First, cleavage of VWF-A2 substrate was measured using plasma from two congenital TTP patients. As shown in FIG. 5A, the ADAMTS-13 deficiency in these two patients was partially restored by mixing the ADAMTS-13-deficient plasma one to one with normal plasma. The assay was performed at 1:1 dilution of plasma. The graph represents the average (×SD) of two different congenital TTP plasmas tested in triplicates. Second, the specific cleavage activity by ADAMTS-13 on the VWF-A2 peptide was also demonstrated using three different dilutions of partially isolated ADAMTS-13 (275 μg/mL of total protein) (FIG. 5B).

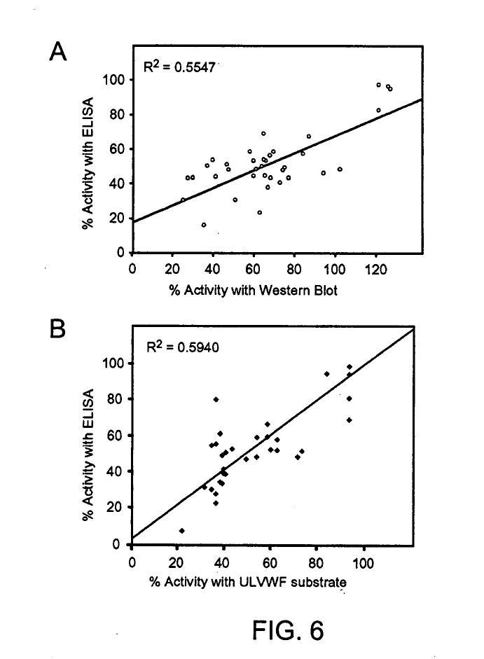

All the samples tested in the assay were concurrently analyzed under static conditions by two immunoblotting methods that analyze ADAMTS-13 cleavage of either ULVWF or recombinant VWF-A2 peptide, and by a flow-based assay that measures ADAMTS-13 cleavage of ULVWF strings secreted by stimulated HUVECs. The enzyme activity determined for the normal plasmas in the ELISA assay (20% plasma or 1:5 dilution) correlated well with the immunoblotting method that used recombinant VWF-A2 as substrate (40% plasma) (FIG. 6A, R2=0.554) and with/the assay that uses ULVWF as the substrate and requires Ba²⁺, or Ca²⁺ and a long (24 h) incubation period (20% plasma) (FIG. 6B, R2=0.594). Although the correlation with the flow based ULVWF multimer string-cleavage assay was less significant among normal plasmas (R2=0.24), severe deficiencies found in plasma from TTP patients were detected in both assays.

Although the present invention and its advantages have been described in detail, it should be understood that various changes, substitutions and alterations can be made herein without departing from the invention as defined by the appended claims. Moreover, the scope of the present application is not intended to be limited to the particular embodiments of the process, machine, manufacture, composition of matter, means, methods and steps described in the specification. As one will readily appreciate from the disclosure, processes, machines, manufacture, compositions of matter, means, methods, or steps, presently existing or later to be developed that perform substantially the same function or achieve substantially the same result as the corresponding embodiments described herein may be utilized. Accordingly, the appended claims are intended to include within their scope such processes, machines, manufacture, compositions of matter, means, methods, or steps.

### REFERENCES

All patents and publications mentioned in the specifications are indicative of the levels of those skilled in the art to which the invention pertains. All patents and publications are herein incorporated by reference to the same extent as if each individual publication was specifically and individually indicated to be incorporated by reference.
U.S. Patent Publication No. 20030073116
International Publication No. WO 02/10437 A2
Aronson D L, Krizek D M, Rick M E. A rapid assay for the vWF protease. Thromb Haemost 2001; 85: 184-5.
Azuma H, Dent J A, Sugimoto M, et al. Independent assembly and secretion of a dimeric adhesive domain of von Willebrand factor containing the glycoprotein Ib-binding site. J Biol Chem 1991; 266: 12342-7.
Bianchi V, Robles R, Alberio L, Furlan M, Lammle B. Von Willebrand factor-cleaving protease (ADAMTS-13) in thrombocytopenic disorders: a severely deficient activity is specific for thrombotic thrombocytopenic purpura. Blood 2002; 100: 710-3.
Bohm M, Vigh T, Scharrer 1. Evaluation and clinical application of a new method for measuring activity of von Willebrand factor-cleaving metalloprotease (ADAMTS-13). Ann Hematol2002; 81: 430-5.
Cruz M A, Handin R I, Wise R J. The interaction of the von Willebrand factor-A1 domain with platelet glycoprotein Ib/IX. The role of glycosylation and disulfide bonding in a monomeric recombinant AII domain protein. J Biol Chem 1993; 268: 21238-45.
Cruz M A, Yuan H, Lee J R, et al. Interaction of the von Willebrand factor (vWF) with collagen. Localization of the primary collagen-binding site by analysis of recombinant vWF a domain polypeptides. J Biol Chem 1995; 270:10822-7.
Cruz M A, Diacovo T G, Emsley J, Mapping the glycoprotein Ib-binding site in the von willebrand factor A1 domain. J Biol Chem 2000; 275:19098-105.
Cruz M A, Whitelock J L, Dong J E Evaluation of ADAMTS-13 activity in plasma using recombinant von Willebrand factor A2 domain polypeptide as substrate. Thromb Haemost 2003; 90: 1234-9.
Dent J A, Berkowitz S O, Ware J, Kasper C K, Ruggeri Z M. Identification of a cleavage site directing the immunochemical detection of molecular abnormalities in type IIA von Willebrand factor. Proc Natl Acad Sci USA 1990; 87: 6306-10.
Dong J F, Monke J L, Nolasco L, Bernardo A, Arceneaux W, Shrimpton C N et al. ADAMTS-13 rapidly cleaves newly secreted ultralarge von Willebrand factor multimers on the endothelial surface under flowing conditions. Blood 2002; 100: 4033-9.
Dong J F, Moake J L, Bernardo A, Fujikawa K, Ball C, Nolasco L et al. Adamts-13 metalloprotease interacts with the endothelial cellderived ultra-large von Willebrand factor. J Bioi Chem 2003; 278: 29633-9.
Estavillo D, Ritchie A, Diacovo T G, et al. Functional analysis of a recombinant glycoprotein Ia/IIa (Integrin alpha(2)beta(1)) I domain that inhibits platelet adhesion to collagen and endothelial matrix under flow conditions. J Biol Chem 1999; 274:35921-6.
Fujikawa K,Suzuki H, McMullen B, Chung D. Purification of human von Willebrandfactor-cleaving protease and its identification as a new member of the metalloproteinase family. Blood 2001; 98: 1662-6.
Gerritsen H E, Robles R, Lammle B, Furlan M. Partial amino acid sequence of purified von Willebrand factor-cleaving protease. Blood 2001; 98; 1654-61.
Furlan M, Robles R, Lammle B. Partial purification and characterization of a protease from human plasma cleaving von Willebrand factor to fragments produced by in vivo proteolysis. Blood 1996; 87: 4223-34
Furlan M, Robles R, Galbusera M, Remuzzi G, Kyrle P A, Brenner B et al. von Willebrand factor-cleaving protease in thrombotic thrombocytopenic purpura and the hemolytic-uremic syndrome. N Engl J Med 1998; 339: 1578-84.
Genitsen H E, Turecek P L, Schwarz H P, Lammle B, Furlan M. Assay of von Willebrand factor (vWF)-cleaving protease based on decreased collagen binding affinity of degraded vWF: a tool for the diagnosis of thrombotic thrombocytopenic purpura (TTP). Thromb Haemost 1999; 82: 1386-9.
He S, Cao H, Magnusson C G, Eriksson-Berg M, Mehrkash M, SchenckGustafsson K et al. Are increased levels of von Willebrand factor in chronic coronary heart disease caused by decrease in von Willebrand factor cleaving protease activity? A study by an immunoassay with antibody against intact bond 842Tyr-843Met of the von Willebrand factor protein. Thromb Res 2001; 103: 241-8.
Jenkins P V, Pasi K J, Perkins S J. Molecular modeling of ligand and mutation sites of the type A domains of human von Willebrand factor and their relevance to von Willebrand's disease. Blood 1998; 91: 2032-44.
Keeney S Cumming A M. The molecular biology of von Willebrand disease. Clin Lab Haematol 2001; 23:209-30.
Kirzek D M, Rick M E. Clinical application of a rapid method using agarose gel electrophoresis and Western blotting to evaluate von ebrand factor protease activity. Electrophoresis 2001; 22: 946-9
Kokame et al. Blood, 15 Jan. 2004, Vol. 103, No. 2, pp. 607-612.
Lankhof H, van Hoeij M, Schiphorst M E, et al. A3 domain is essential for interaction of von Willebrand factor with collagen type III. Thromb Haemost 1996; 75:950-8.
Lankhof H, Damas C, Schiphorst M E, Ijsseldijk M J, Bracke M, Furlan M et al. von Willebrand factOr without the A2 domain is resistant to proteolysis. ThrombBaernost 1997; 77: 1008-13.
Loof A H, van Vliet H H, Kappers-Klunne M C Low activity of von Willebrand factor-cleaving protease is not restricted to patients suffering from thrombotic thrombocytopenic purpura. Br J Haematol 2001; 112: 1087-8.
Mannucci P M, Canciani M T, Forza I, Lussana F, Lattuada A, Rossi E. Changes in health and disease of the metalloprotease that cleaves von Willebrand factor. Blood 2001; 98: 2730-5.
Moake J L, Rudy C K, Troll J H, Weinstein M J, Colannino N M, Azocar J et al. Unusually large plasma factor VIII:von Willebrand factor multi-[Q7] mers in chronic relapsing thrombotic thrombocytopenic purpura. N Engl J Med 1982; 307: 1432-5.
Moake J L, Rudy C K, Troll J H, Schafer A I, Weinstein M J, Colannino N M et al. Therapy of chronic relapsing thrombotic thrombocytopenic purpura with prednisone and azathioprine. Am J Hematol 1985; 20: 73-9.
Moake J, Chintagumpala M, Turner N, McPherson P, Nolasco L, Steuber C et al. Solvent/detergent-treated plasma suppresses shearinduced platelet aggregation and prevents episodes of thrombotic thrombocytopenic purpura. Blood 1994; 84: 490-7.
Moake J L. Thrombotic microangiopathies. N Engl J Med 2002; 347: 589-600.
Obert B, Tout H, Veyradier A, Fressinaud E, Meyer D, Girma J P. Estimation of the von Willebrand factor-cleaving protease in plasma using monoclonal antibodies to vWF. Thromb Haemost 1999; 82: 1382-5.
Shenkman B, Inbal A, Tamarin I, Lubetsky A, Savion N, Varon D. Diagnosis of thrombotic thrombocytopenic purpura based on modulation by patient plasma of normal platelet adhesion under flow condition. Br J Haematol 2003; 120: 597-604.
Sugimoto M, Ricca G, Hrinda M E, et al. Functional modulation of the isolated glycoprotein Ib binding domain of von Willebrand factor expressed in Escherichia coli. Biochemistry 1991; 30; 5202-9.
Tsai H M, Sussman I I, Nagel R L. Shear stress enhances the proteolysis von Willebrand factor in normal plasma. Blood 1994; 83: 2171-9
Tsai H M. Physiologic cleavage of von Willebrand factor by a plasma protease is dependent on its conformation and requires calcium ion. Blood 1996; 87: 4235-44.
Tsai H M, Lian E C Antibodies to von Willebrand factor-cleaving protease in acute thrombotic thrombocytopenic purpura. N Engl J Med 1998; 339: 1585-94.
Tsai H M. Deficiency of ADAMTS 13 and thrombotic thrombocytopenic PlJrPura. Blood 2002; 100: 3839-40.
Zheng X, Chung D, Takayama T K, Majerus E M, Sadler J E, Fujikawa K. Structure of von Willebrand factor-cleaving protease (ADAMTS 13), a metalloprqtease involved in thrombotic thrombocytopenic purpura. J Bioi Chern 2001; 276: 41059-63.
Zimmerman T S, Dent J A, Ruggeri Z M, Nannini L H. Subunit composiotion of plasma von Willebrand factor. Cleavage is present in normal individuals, increased in IIA and IIB von Willebrand disease, but minimal in variants with aberrant structure of individual oligomers (types IIC, IID; and IIE). J Clin Invest 1986; 77: 947-51.

1. A method for detecting ADAMTS-13 activity in a biological sample comprising the steps of:
collecting the biological sample;
contacting the sample with a recombinant polypeptide comprising a functional ADAMTS-13 cleavage site; and
measuring cleavage of said recombinant polypeptide, wherein the presence of cleaved recombinant polypeptide indicates ADAMTS-13 activity.

2. The method of claim 1, wherein measuring cleavage of said recombinant polypeptide comprises gel electrophoresis or immunoblotting.

3. The method of claim 1, wherein measuring cleavage of said recombinant polypeptide comprises an ELISA.

4. The method of claim 1, wherein the recombinant polypeptide comprises the A2 domain of von Willebrand factor.

5. The method of claim 1, wherein the functional ADAMTS-13 cleavage site comprises the ADAMTS-13 cleavage site of von Willebrand factor.

6. The method of claim 1, wherein the recombinant polypeptide comprises SEQ ID NO:1

7. The method of claim 1, wherein the recombinant polypeptide comprises at least one epitope tag.

8. The method of claim 7, wherein the epitope tag is selected from the group consisting of histidine tag, tag-100, c-myc, FLAG, E2, HA and GST.

9. The method of claim 7, wherein the recombinant polypeptide further comprises a fluorescent tag.

10. The method of claim 1, wherein said contacting takes place under non-denaturing conditions.

11. The method of claim 1, wherein said contacting is for a period of time of less than about 2 hours.

12. The method of claim 1, wherein said contacting is for a period of time of less than about 1 hours.

13. The method of claim 1, wherein the contacting and measuring steps do not require the addition of ions.

14. A method of diagnosing a disease in a mammal associated with decreased ADAMTS-13 activity comprising the steps of:
collecting a biological sample from the mammal;
contacting the sample with a recombinant polypeptide comprising a functional ADAMTS-13 cleavage site; and
measuring cleavage of said recombinant polypeptide, wherein a decreased level of cleaved recombinant polypeptide in the sample as compared to a control sample is diagnostic for the disease.

15. The method of claim 14, wherein the disease associated with decreased ADAMTS-13 activity is selected from the group consisting of TTP, DIC, sepsis, heparin-induced thrombocytopenia, thrombotic microangiopathy, cancer, and liver disease.

16. A method of identifying a mammal at risk of developing TTP disease comprising the steps of:
collecting a biological sample from the mammal;
contacting the sample with a recombinant polypeptide comprising a functional ADAMTS-13 cleavage site; and
measuring cleavage of said recombinant polypeptide, wherein a decreased level of cleaved recombinant polypeptide in the sample as compared to a control sample indicates that the mammal is at risk of developing TTP.

17. A recombinant polypeptide comprising a functional ADAMTS-13 cleavage site and at least one epitope tag, wherein the at least one epitope tag is located on the N-terminal side or the C-terminal side of the ADAMTS-13 cleavage site.

18. The recombinant polypeptide of claim 17, wherein the recombinant polypeptide comprises SEQ ID NO:1.

19. The recombinant polypeptide of claim 17, further comprising a second epitope tag, wherein the second epitope tag is located on a side opposite the ADAMTS-13 cleavage site of the at least one epitope tag.

20. The recombinant polypeptide of claim 19, wherein the at least one epitope tag is selected from the group consisting of histidine tag, tag-100, c-myc, FLAG, E2, HA, and GST.

21. The recombinant polypeptide of claim 19, wherein the second epitope tag is selected from the group consisting of histidine tag, tag-100, c-myc, FLAG, E2, and HA, and is different than the at least one epitope tag.

22. The recombinant polypeptide of claim 19, wherein the at least one epitope tag is selected from the group consisting of histidine tag, tag-100, c-myc, FLAG, E2, HA, and GST, wherein the second epitope tag is selected from the group consisting of histidine tag, tag-100, c-myc, FLAG, E2, and HA, and wherein the second epitope tag is different than the at least one epitope tag.

23. A host cell expressing the recombinant polypeptide of claim 17.

24. An isolated nucleic acid encoding the recombinant polypeptide of claim 17.

25. A kit comprising the polypeptide of claim 17, an antibody specific for the first epitope tag, and an antibody specific for the second epitope tag.

26. The kit of claim 25, further comprising a conjugated antibody.

27. The kit of claim 26, wherein the conjugated antibody comprises a peroxidase or FITC.

28. The recombinant polypeptide of claim 25, further comprising at least one epitope tag.
